(19) United States
(12) Patent Application Publication
(10) Pub. No.: US 2026/0010026 A1
(43) Pub. Date: Jan. 8, 2026

(54) ENGINEERED LIQUID CRYSTAL SHUTTER AS A DYNAMIC LONG-PASS OPTICAL FILTER

(71) Applicant: Blaze Bioscience, Inc., Seattle, WA (US)

(72) Inventors: Mark Schnittker, Sunnyvale, CA (US); David Kittle, Cherryville (CA)

(21) Appl. No.: 18/992,474

(22) PCT Filed: Jul. 7, 2023
(86) PCT No.: PCT/US2023/069756

§ 371 Date: Jan. 8, 2025

Related U.S. Application Data
(60) Provisional application No. 63/359,535, filed on Jul. 8, 2022.

(51) Int. Cl. G02F 1/133; G01J 3/02; G01J 3/12; G01J 3/28; G01J 3/44; G01N 21/64; G01N 33/483; G02B 21/00; G02B 21/36; G02F 1/1333; G02F 1/1335; G02F 1/139; G03B 9/08

(52) U.S. Cl. CPC G02F 1/13306; G01J 3/0224; G01J 3/0232; G01J 3/2823; G01J 3/4406; G01N 21/6458; G01N 21/6486; G01N 33/4833; G02F 1/133382; G02F 1/133533; G02F 1/1396; G01J 2003/1213; G01J 2003/2826; G01N 2021/6471; G01N 2201/067; G02B 21/0076; G02B 21/361; G02F 2203/026; G02F 2203/055; G02F 2203/11; G03B 9/08

(57) ABSTRACT

The present disclosure relates to a novel liquid crystal assembly for use in multispectral imaging systems. In particular, the liquid crystal assembly includes an engineered polarizer and a liquid crystal. In various aspects, the liquid crystal assembly takes advantages of short-comings found in conventional polarizers and liquid crystals requiring complex temperature regulation.

### FIELD OF DISCLOSURE

The present disclosure generally relates to an engineered liquid crystal shutter for use in multi-spectral imaging. In particular, the shutter relies on previously undesirable limitations of optical polarizers to increase the ratio of transmitted light available for imaging and analysis at video rate switching speeds to one or more optical sensors.

### BACKGROUND

Multi-color or “Multispectral” imaging is a method that surveys information from a broad range of different wavelengths of light and displays them into the relatively narrow visible range. When processing spectral information, it is often necessary to apply different image analysis techniques to the different wavelengths of light to properly weigh their values before recombining. To allow independent processing of the different color images, the spectrum is commonly broken into multiple spectral bands and the images from those spectral bands are collected independently. The hardware used to image the broad spectrum may include multiple image sensors that are responsible to collect their respective spectral bands.

Liquid crystal shutters are commonly used in liquid crystal displays as well as to enable video rate switching for Digital Light Projection (DLP) systems, for example, in movie theaters. Typically, when linear polarizers are used for liquid crystal shutters, the spectral range of effective shuttering is greatly limited by the polarizing extinction ratio of the polarizing filters and the limited transmission of the liquid crystal itself.

### SUMMARY

The present disclosure generally relates to a liquid crystal assembly for use with an imaging system having a light source. The assembly includes a polarizer disposed in a path of light from the light source. The polarizer is configured to have a light cut-off in a range outside of its range of polarization. The assembly also includes a liquid crystal disposed in a path of light transmitted through the polarizer and a drive voltage source configured to apply a first voltage and a second voltage to the liquid crystal. The first voltage and the second voltage achieve high and low electric fields across the liquid crystal. The first voltage and the second voltage toggle the liquid crystal between an open state and a closed state. In one aspect, applying one of the first voltage or second voltage toggles the liquid crystal to the open state. Applying one of the first voltage or the second voltage toggles the liquid crystal to the closed state.

In various aspects, the polarizer is configured to be transparent above the light cut-off. In one aspect, the light cut-off includes a longest wavelength of a spectral range and the polarizer is configured to be ineffective and transparent above the longest wavelength. In some aspects, the liquid crystal includes a Twisted Nematic (TN) cell, Pi cell, thermotropic liquid crystal, and lyotropic liquid crystal, liquid crystal display (LCD), liquid crystal with photoconductor properties, or liquid crystal receptive to development and tuning under an electric field. In some aspects, the second voltage induces a backflow in the liquid crystal.

In various aspects, one of the first voltage or second voltage is 0 V. In another aspect, the second voltage is a plurality of voltages comprising a bi-polar square wave with an average value of 0 V. In an aspect, the first voltage, or the second voltage, or both the first voltage and the second voltage, includes a plurality of voltages creating a bi-polar square wave with an average value of 0 V. In an another aspect, the bi-polar square wave includes an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive. In a further aspect, the second voltage is a bi-polar square wave having an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

In various aspects, a bi-polar square wave amplitude is modulated at a video capture rate for the imaging system. In another aspect, the amplitude of the bi-polar square wave is modulated from 500 to 2000 Hz. In some aspects, the drive voltage source toggles the liquid crystal between the first voltage and the second voltage in durations from 1 milliseconds to 40 milliseconds inclusive, or 2 milliseconds to 60 milliseconds inclusive, 60 milliseconds to 100 milliseconds inclusive, and up to 300 milliseconds inclusive. In some aspects, the drive voltage source toggles the liquid crystal between the first voltage and second voltage at a video rate sampling duration of approximately between 1 ms to 10 ms, between 2 ms and 20 ms, or 6 ms.

In various aspects, the liquid crystal is a twisted nematic (TN) liquid crystal. In an aspect, the liquid crystal is heated to a range of 30° C. to 50° C. In another aspect, the liquid crystal is enclosed in a housing of the imaging system and the liquid crystal is heated by waste heat generated by other components of the imaging system. In an aspect, a transition speed between the open state and the closed state is determined by a temperature of the liquid crystal and a voltage source drive level.

In various aspects, the polarizer is an engineered dyestuff polarizer. In an aspect, the polarizer is configured to have a light cut-off in a range optimal for a fluorescent imaging process. In some aspects, the polarizer is chosen to lose effectiveness or cutoff between 650 and 800 nm, or 600 nm and about 800 nm, or between about 700 nm to about 800 nm, about 800 nm to about 950 nm, about 800 nm to about 880 nm, about 775 nm to about 795 nm, or about 785 nm. In another aspect, the polarizer is chosen to lose effectiveness or cutoff by 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm.

In various aspects, further includes a beam splitter. In an aspect, the beam splitter has a front surface and a back surface. In some aspects, the front surface has a front surface coating and the back surface has a back surface coating. In another aspect, the front surface coating has a higher P polarization reflection than the back surface coating. In an aspect, the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1. In an aspect, S polarized reflections of the front surface and the back surface of the beam splitter are minimized by the front surface coating and the back surface coating. In another aspect, the polarizer is configured to block or attenuate ghosting or secondary reflections reflected from a beam splitter. In an aspect, the polarizer is configured to block S polarized light from the beam splitter. In another aspect, the polarizer is configured to allow P polarized light to pass through the liquid crystal assembly to an imaging lens and/or a camera when the liquid crystal is in the open state. In an aspect, the first polarizer allows a front to back reflectivity ratio from the beam splitter of at least 2:1, 4:1, 6:1, 8:1, 10:1, 12:1, 15:1, 17:1, or 20:1 to pass through to the imaging lens and/or camera. In some aspects, a total reflectivity from the beam splitter is about 11% or lower, about 10% or lower, about 9% or lower, about 8% or lower, about 7% or lower, about 6% or lower, about 5% or lower, about 4% or lower, or about 3% or lower.

In various aspects, the liquid crystal assembly includes two polarizers or more. In another aspect, the liquid crystal assembly is adapted to a medical device including a surgical microscope, confocal microscope, fluorescence scope, exoscope, endoscope, or surgical robot. In another aspect, the liquid crystal assembly is adapted to the medical device optionally through use of an optical gasket. In some aspects, the medical device is a KINEVO system (e.g., KINEVO 900), OMPI PENTERO system (e.g., PENTERO 900, PENTERO 800), or Leica FL800 system. In another aspect, the liquid crystal assembly is used in a method for imaging an emission light emitted by a fluorophore.

The present disclosure also generally relates to a method for imaging an emission light emitted by a fluorophore at an imaging system including a liquid crystal assembly. The method includes allowing or directing a visible light to a sample, directing an excitation light to the sample, and directing the emission light and a reflected visible light from the sample to the liquid crystal assembly. In various aspects of the aforementioned method, the liquid crystal assembly includes an engineered polarizer disposed in a path of light from sample; the polarizer configured to have a light cut-off in a range between 600 nm and 900 nm. The liquid crystal assembly also includes a liquid crystal disposed in a path of light transmitted through the polarizer and a drive voltage source configured to apply a first voltage and a second voltage to the liquid crystal; wherein the first voltage and the second voltage toggle the liquid crystal between an open state and a closed state. The method further includes directing the emission light and the reflected visible light through the engineered polarizer, wherein the reflected visible light having a wavelength below the light cut-off passes through the polarizer with approximately 50% attenuation and the emission light having a wavelength above the light cut-off passes through the polarizer with minimal attenuation. The method further includes directing a polarized visible light and unpolarized emission light to the liquid crystal, applying the first voltage to the liquid crystal to assume the open state for a first video rate sampling duration, and detecting the polarized light at an imaging sensor. The method further includes applying the second voltage to the liquid crystal to assume the closed state and block the polarized light from passing to the imaging sensor, and detecting the unpolarized emission light at the imaging sensor during the closed state for a second video rate sampling duration.

In various aspects of the disclosed methods, the minimal attenuation of the emission light having a wavelength above the light cut-off is 0% attenuation to 15% attenuation. In one aspect, the reflected visible light having a wavelength below the light cut-off passes through the polarizer at approximately 50% or less attenuation. In another aspect, the first video rate sampling duration is between 1 ms to 10 ms, between 2 ms and 20 ms, or 6 ms. In some aspect, the second video rate sampling duration is between 1 ms to 10 ms, between 2 ms and 20 ms, or 6 ms. In some aspects, the first voltage and second voltage achieve high and low electric fields respectively across the liquid crystal.

In various aspects of the disclosed methods, the first voltage and the second voltage toggle the liquid crystal between an open state and a closed state. In an aspect, the first voltage toggles the liquid crystal to the open state and the second voltage toggles the liquid crystal to the closed state, or vice versa. In another aspect, one of the first voltage or the second voltage is 0 V. In an aspect, the second voltage is a plurality of voltages comprising a bi-polar square wave with an average value of 0 V. In another aspect, the first voltage, or the second voltage, or both the first voltage and the second voltage, includes a plurality of voltages creating a bi-polar square wave with an average value of 0 V. In an aspect, the bi-polar square wave has an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive. In another aspect, the second voltage is a bi-polar square wave having an amplitude of +/−0.1 to +/−5V p-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive. In some aspects, a bi-polar square wave amplitude is modulated at a video capture rate for the imaging system. In another aspect, the first voltage is a bi-polar square wave having an amplitude of +/−0.1 to +/−5 Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

In various aspects of the disclosed methods, the amplitude is modulated from 500 to 2000 Hz. According to various aspects, the disclosed methods further include heating the liquid crystal using waste heat from the imaging system. In some aspects, the polarizer is a dyestuff polarizer. In another aspect, the polarizer is configured to have a light cut-off in a range optimal for a fluorescent imaging process. In various aspects, the polarizer in the liquid crystal assembly is configured to have a light cut-off in a range between about 600 nm and about 850 nm, or between about 700 nm to about 850 nm, about 800 nm to about 950 nm, about 800 nm to about 880 nm, about 775 nm to about 795 nm, or about 785 nm. In another aspect, the polarizer in the liquid crystal assembly is chosen to lose effectiveness or cut off by 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm. In other aspects, the polarizer in the liquid crystal assembly further includes two polarizers or more.

According to various aspects of the disclosed methods, the excitation light is engineered to enhance a signal to noise (SNR) of an NIR image such that a peak intensity and duration of the excitation light creates a controlled energy excitation that falls below a safety threshold. According to various aspects, the disclosed methods further include directing the emission light and the reflected visible light to a beam splitter prior to directing the emission light and the reflected light to the liquid crystal assembly. In an aspect, the beam splitter has a front surface and a back surface. In another aspect, the front surface has a front surface coating and the back surface has a back surface coating. In an aspect, the front surface coating has a higher P polarization reflection than the back surface coating. In another aspect, the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1. In another aspect, S polarized reflections of the front surface and the back surface of the beam splitter are minimized by the front surface coating and the back surface coating. In a further aspect, the polarizer is configured to block or attenuate ghosting or secondary reflections reflected from a beam splitter. In an aspect, the first polarizer is configured to block S polarized light from the beam splitter. In another aspect, the polarizer is configured to allow P polarized light to pass through the liquid crystal assembly to an imaging lens or a camera when the liquid crystal is in the open state. In an aspect, the first polarizer allows a front to back reflectivity ratio from the beam splitter of at least 2:1, 4:1, 6:1, 8:1, 10:1, 12:1, 15:1, 17:1, or 20:1 to pass through to an imaging lens and camera. In some aspects, a total reflectivity from the beam splitter is about 11% or lower, about 10% or lower, about 9% or lower, about 8% or lower, about 7% or lower, about 6% or lower, about 5% or lower, about 4% or lower, or about 3% or lower.

The present disclosure also relates to a liquid crystal assembly including any feature described, either individually or in combination with any other features, in any configuration, as disclosed herein.

The present disclosure also relates to method for imaging an emission light emitted by a fluorophore at an imaging system including a liquid crystal assembly comprising any feature described, either individually or in combination with any other features, in any configuration, as disclosed herein.

The present disclosure also relates to a liquid crystal assembly for use with an imaging system having a light source to provide an excitation light. The liquid crystal assembly includes a first polarizer disposed in a path of light from the light source; the first polarizer configured to have a light cut-off in a range outside of its range of polarization, a liquid crystal disposed in a path of light transmitted through the first polarizer, and a second polarizer disposed in a path of light transmitted through the liquid crystal. The second polarizer is configured to have a light cut-off in a range outside of its range of polarization. The liquid crystal assembly also includes a drive voltage source configured to apply a first voltage and a second voltage to the liquid crystal. The first voltage and second voltage toggle the liquid crystal between an open state and a closed state.

In various aspects, the first polarizer and the second polarizer are cross-polarized. In an aspect, the liquid crystal is in the open state when one of the first voltage or second voltage is applied to the liquid crystal. In another aspect, one of the first voltage or second voltage is 0 V. In some aspects, the liquid crystal is in the closed state when one of the first voltage or the second voltage is applied to the liquid crystal. In another aspect, the second voltage is a bi-polar square wave. In an aspect, the bi-polar square wave has an amplitude of +/−0.1 to +/−5Vp-p, +/−3 to +/−30Vp-p, +/−16 Vp-p, +/−24 Vp-p, or +/−30 Vp-p.

In various aspects, the liquid crystal assembly further includes an imaging lens in a path of light transmitted through the second polarizer, and a camera in a path of light transmitted through the imaging lens. In another aspect, when the liquid crystal is in the open state the liquid crystal rotates the polarization of a polarized visible light transmitted through the first polarizer allowing the polarized visible light to pass through the second polarizer to the imaging lens at about 50% or less attenuation. In some aspects, when the liquid crystal is in the closed state the liquid crystal does not rotate the polarization of the polarized visible light and the polarized visible light transmitted through the first polarizer is blocked by the second polarizer.

In various aspects, the light cut-off range of the first polarizer and the second polarizer is where a polarizer extinction ratio is poor or minimal. In another aspect, light outside the light cut-off range of the first polarizer and second polarizer passes through the first polarizer and the second polarizer to the imaging lens with no attenuation or a minimal attenuation. In an aspect, the minimal attenuation is 15% or less. In some aspects, the light cut-off range of the first polarizer is about 700 nm to about 800 nm or about 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm. In another aspect, the light cut-off range of the second polarizer is about 700 nm to about 800 nm or about 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm. In another aspect, the light cut-off range of the first polarizer and the second polarizer is above a longest wavelength of the visible light spectrum.

According to various aspects, when the liquid crystal is in the open state the liquid crystal assembly is configured to view visible light. In another aspect, when the liquid crystal is in the closed state the liquid crystal assembly is configured to block visible light. In an aspect, the blocked visible light allows for fluorescent imaging from a fluorophore. In some aspects, the fluorophore provides an emission light emitted by the fluorophore. In an aspect, the fluorophore is provided an excitation light. In some aspects, the excitation light is white light, NIR light, IR light, or any other type of excitation light. In an aspect, the excitation light is a near infrared light provided by a laser diode.

In various aspects, the liquid crystal assembly further includes a beam splitter. In some aspects, the beam splitter has a front surface and a back surface. In an aspect, the front surface has a front surface coating and the back surface has a back surface coating. In some aspects, the front surface coating has a higher P polarization reflection than the back surface coating. In another aspect, the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1. In an aspect, S polarized reflections of the front surface and the back surface of the beam splitter are minimized by the front surface coating and the back surface coating. In another aspect, the first polarizer is configured to block or attenuate ghosting or secondary reflections reflected from a beam splitter. In some aspects, the first polarizer is configured to block S polarized light from the beam splitter. In another aspect, the first polarizer and the second polarizer are configured to allow P polarized light to pass through the liquid crystal assembly to an imaging lens or a camera when the liquid crystal is in the open state. In an aspect, the first polarizer allows a front to back reflectivity ratio from the beam splitter of at least 2:1, 4:1, 6:1, 8:1, 10:1, 12:1, 15:1, 17:1, or 20:1 to pass through to an imaging lens and a camera. In another aspect, a total reflectivity from the beam splitter is about 11% or lower, about 10% or lower, about 9% or lower, about 8% or lower, about 7% or lower, about 6% or lower, about 5% or lower, about 4% or lower, or about 3% or lower.

In various aspects, the imaging lens and camera are configured to view a fluorescent image using a video rate sampling duration of between 1 ms to 10 ms, between 2 ms and 20 ms, or 6 ms. In an aspect, the fluorescent image has a reduced motion blur.

The present disclosure also relates to a method for imaging an emission light emitted by a fluorophore at an imaging system comprising a liquid crystal assembly. The method includes allowing or directing a visible light to a sample, directing an excitation light to the sample, and directing the emission light and a reflected visible light to the liquid crystal assembly. The liquid crystal assembly includes a first engineered polarizer in a path of light from the sample. The first engineered polarizer is configured to have a light cut-off in a range between 600 nm and 900 nm or 800 nm to 880 nm. The liquid crystal assembly also includes a liquid crystal disposed in a path of light transmitted through the first engineered polarizer. The liquid crystal assembly also includes a second engineered polarizer in a path of light transmitted through the liquid crystal. The second engineered polarizer is configured to have a light cut-off in a range between 600 nm and 900 nm or 800 nm to 880 nm. In an aspect, the first engineered polarizer and second engineered polarizer are cross-polarized. The liquid crystal assembly also includes a drive voltage source configured to apply a first voltage and a second voltage to the liquid crystal; wherein the first voltage and the second voltage toggle the liquid crystal between an open state and a closed state. In an aspect, the disclosed method also includes directing the emission light and the reflected visible light through the first engineered polarizer, wherein the first engineered polarizer polarizes the reflected visible light below the light cut-off range producing a polarized reflected visible light. The disclosed method further includes directing the emission light and the polarized reflected visible light through the liquid crystal, applying the first voltage to the liquid crystal to assume the open state for a first video rate sampling duration, and rotating the polarization of the polarized reflected visible light using the liquid crystal in the open state. The disclosed method further includes directing the emission light and the polarized reflected visible light through the second engineered polarizer, wherein the second engineered polarizer allows the polarized reflected visible light to pass through the second engineered polarizer. The disclosed method further includes detecting a first portion of the polarized reflected visible light passing through the second engineered polarizer during the open state at an imaging sensor. The disclosed method further includes applying the second voltage to the liquid crystal to assume the closed state for a second video rate sampling duration, passing the emission light and the polarized reflected visible light through the liquid crystal without rotating the emission light and the polarized reflected visible light, blocking the polarized reflected visible light with the second engineered polarizer and detecting a portion of the emission light during the closed state at the imaging sensor.

In various aspects of the disclosed method, the method further includes blocking a secondary reflection of the reflected visible light using the first polarizer. In an aspect, the excitation light is provided to the sample via a laser diode.

In various aspects of the disclosed method, the method further includes turning the laser diode off to stop providing an excitation light, providing the second voltage to the liquid crystal to assume the closed state for a third video rate sampling duration, detecting a dark background image of the sample, and subtracting the dark background image of the sample from the detected portion of the emission light to produce a fluorescent image.

In various aspects of the disclosed method, the first video rate sampling duration is about 2 ms to about 10 ms. In an aspect, the second video rate sampling duration is about 8 ms to about 16 ms. In another aspect, the third video rate sampling duration is about 8 ms to about 16 ms.

In various aspects of the disclosed method, one of the first voltage or the second voltage is about 0 V. In another aspect, the second voltage is a bi-polar square wave. In an aspect, bi-polar square wave has an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

In various aspects of the disclosed method, the fluorescent image has a reduced motion blur. In an aspect, the second video rate sampling duration and third video rate sampling duration allow a post processing by digital gain. In another aspect of the disclosed method, the disclosed method further includes orienting the first engineered polarizer to block an undesired polarized light. In an aspect, second video rate sampling duration and third video rate sampling duration enhance a signal to noise ratio (SNR) and a contrast to noise ratio (CNR) of the fluorescent image. In an aspect, the enhanced CNR enables a fast frame rate. In another aspect, the disclosed method provides real-time viewing of the reflected visible light and a fluorescent image.

In various aspects of the disclosed method, the disclosed method further includes directing the emission light and the reflected light to a beam splitter prior to directing the emission light and the reflected light to the liquid crystal assembly. In an aspect, the beam splitter has a front surface and a back surface. In another aspect, the front surface has a front surface coating, and the back surface has a back surface coating. In some aspects, the front surface coating has a higher P polarization reflection than the back surface coating. In an aspect, the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1. In another aspect, S polarized reflections of the front surface and the back surface of the beam splitter are minimized by the front surface coating and the back surface coating. In another aspect, the first polarizer is configured to block or attenuate ghosting or secondary reflections reflected from a beam splitter. In an aspect, the first polarizer is configured to block S polarized light from the beam splitter. In another aspect, the first polarizer and the second polarizer are configured to allow P polarized light to pass through the liquid crystal assembly to an imaging lens and/or a camera when the liquid crystal is in the open state. In some aspects, the first polarizer allows a front to back reflectivity ratio from the beam splitter of at least 2:1, 4:1, 6:1, 8:1, 10:1, 12:1, 15:1, 17:1, or 20:1 to pass through to an imaging lens and a camera. In various aspects, a total reflectivity from the beam splitter is about 11% or lower, about 10% or lower, about 9% or lower, about 8% or lower, about 7% or lower, about 6% or lower, about 5% or lower, about 4% or lower, or about 3% or lower.

The present disclosure also relates to a method of imaging an abnormal tissue, cancer, tumor, vasculature or structure in a sample from a subject, the method includes producing an image of the vasculature or structure by imaging fluorescence using the liquid crystal assemblies disclosed herein.

The present disclosure also relates to a method of imaging an abnormal tissue, cancer, tumor, vasculature or structure in a sample from a subject in accordance with any of the methods disclosed herein, the method includes producing an image of the abnormal tissue, cancer, tumor, vasculature or structure by imaging fluorescence using an imaging system comprising a liquid crystal assembly.

In various aspects, the fluorescence imaged is autofluorescence, a contrast or imaging agent, chemical agent, a radiolabel agent, radiosensitizing agent, photosensitizing agent, fluorophore, therapeutic agent, an imaging agent, a diagnostic agent, a protein, a peptide, a nanoparticle, or a small molecule, or any combination thereof or any combination thereof. In an aspect, the fluorescence imaged is autofluorescence, a contrast or imaging agent, chemical agent, a radiolabel agent, radiosensitizing agent, photosensitizing agent, fluorophore, therapeutic agent, an imaging agent, a diagnostic agent, a protein, a peptide, a nanoparticle, or a small molecule, or any combination thereof. In an aspect of the disclosed methods, the method further includes administering a contrast or imaging agent to the subject.

The present disclosure also relates to a method of imaging an abnormal tissue, cancer, tumor, vasculature or structure in a fluorophore from a subject using the any of the disclosed liquid crystal assemblies. The method includes administering a contrast or imaging agent to the subject, and producing an image of the abnormal tissue, cancer, tumor, vasculature or structure by imaging the contrast or imaging agent using an imaging system.

The present disclosure also relates to a method of imaging an abnormal tissue, cancer, tumor, vasculature or structure in a fluorophore from a subject in accordance with any of the disclosed methods. The disclosed methods further include administering a contrast or imaging agent to the subject, and producing an image of the abnormal tissue, cancer, tumor, vasculature or structure by imaging the contrast or imaging agent using an imaging system. In an aspect, the contrast or imaging agent includes a dye, a fluorophore, a fluorescent biotin compound, a luminescent compound, a chemiluminescent compound, or any combination thereof.

In various aspects of the disclosed methods, the contrast or imaging agent further includes a protein, peptide, amino acid, nucleotide, polynucleotide, or any combination thereof. In an aspect, the contrast or imaging agent further includes tozuleristide. In another aspect, the contrast or imaging agent absorbs a wavelength between from about 200 nm to about 900 nm. In other aspects, the contrast or imaging agent includes DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, rythrosine, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin, coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl(2-aminoethyl) sulfonamide), a bimane dye, bimane mercaptoacetic acid, an NBD dye, a QsY 35, or any combination thereof.

In various aspects of the disclosed methods, administering includes intravenous administration, intramuscular administration, subcutaneous administration, intraocular administration, intra-arterial administration, peritoneal administration, intratumoral administration, intradermal administration, or any combination thereof. In an aspect, the imaging includes tissue imaging, ex vivo imaging, intraoperative imaging, or any combination thereof. In an aspect, the sample is in an in vivo sample, an in situ sample, an ex vivo sample, or an intraoperative sample. In another aspect, the sample is an organ, an organ substructure, a tissue, or a cell. In various aspect, the sample autofluoresces. In an aspect, autofluorescence of the sample includes an ocular fluorophore, tryptophan, or protein present in a tumor or malignancy. In an aspect, the method is used to visualize vessel flow or vessel patency.

In various aspects of the disclosed method, the abnormal tissue, cancer, tumor, vasculature or structure includes a blood vessel, lymph vasculature, neuronal vasculature, or CNS structure. In an aspect, the imaging is angiography, arteriography, lymphography, or cholangiography. In another aspect, the imaging includes detecting a vascular abnormality, vascular malformation, vascular lesion, organ or organ substructure, cancer or diseased region, tissue, structure or cell. In another aspect, the vascular abnormality, vascular malformation, or vascular lesion is an aneurysm, an arteriovenous malformation, a cavernous malformation, a venous malformation, a lymphatic malformation, a capillary telangiectasia, a mixed vascular malformation, a spinal dural arteriovenous fistula, or a combination thereof. In an aspect, an organ or organ substructure is brain, heart, lung, kidney, liver, or pancreas.

In various aspects of the disclosed methods, the disclosed methods further include performing surgery on the subject. In an aspect, the surgery includes angioplasty, cardiovascular surgery, aneurysm repair, valve replacement, aneurysm surgery, arteriovenous malformation or cavernous malformation surgery, venous malformation surgery, lymphatic malformation surgery, capillary telangiectasia surgery, mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or diseased region, tissue, structure or cell of the subject, or any combination thereof.

In various aspects of the disclosed methods, the imaging includes imaging a vascular abnormality, cancer or diseased region, tissue, structure, or cell of the subject after surgery. In various aspects, the disclosed methods further include treating a cancer in the subject. In an aspect, the disclosed methods further include repair of an intracranial CNS vascular defect, a spinal CNS vascular defect, peripheral vascular defects, removal of abnormally vascularized tissue, ocular imaging and repair, anastomosis, reconstructive or plastic surgery, plaque ablation or treatment or restenosis in atherosclerosis, repair or resection (including selective resection), preservation (including selective preservation), of vital organs or structures such as nerves, kidney, thyroid, parathyroid, liver segments, or ureters, identification and management (sometimes preservation, sometimes selective resection) during surgery; diagnosis and treatment of ischemia in extremities, or treatment of chronic wounds.

### BRIEF DESCRIPTION OF FIGURES

FIG. 1 is a diagram of a liquid crystal shutter assembly shown with a camera, according to one aspect.

FIG. 2 illustrates a spectral response of an exemplary camera, according to one aspect.

FIG. 3 illustrates a spectral response of an exemplary camera to light attenuated by a long-pass filter, according to one aspect.

FIG. 4 illustrates changes in the extinction ratio of light for a liquid crystal cell in an open state and a closed state, according to one aspect.

FIG. 5 illustrates closed state transmission ratios for a liquid crystal cell in conjunction with various polarizers, according to one aspect.

FIG. 6 illustrates liquid crystal cell transition speeds in response to different applied voltages, according to one aspect.

FIG. 7 illustrates liquid crystal cell transition speeds at different temperatures, according to one aspect.

FIG. 8 illustrates optical blocking in a liquid crystal cell in response to an applied voltage, according to one aspect.

FIG. 9 illustrates the average power imparted by a pulsed or controlled excitation energy imparted to a tissue sample compared against the average power of an exemplary clinical level of energy imparted to the tissue sample, according to one aspect.

FIG. 10 illustrates example signal-to-noise ratios obtained using a pulsed or controlled excitation light source against an exemplary clinical laser excitation light, according to one aspect.

FIG. 11 illustrates a liquid crystal shutter assembly, according to one aspect.

FIG. 12 illustrates transmission ratios for a liquid crystal media in an open state and a closed state, according to one aspect.

FIG. 13 illustrates a diagram of a liquid crystal shutter assembly shown with a camera, according to one aspect.

FIG. 14A illustrates a visible light image taken with a liquid crystal shutter assembly in an open state, according to one aspect.

FIG. 14B illustrates a visible light image taken with a liquid crystal shutter assembly in a closed state, according to one aspect.

FIG. 15 illustrates contrast to noise ratio using drive voltages supplied to the liquid crystal assembly, according to one aspect.

FIG. 16 illustrates exposure rates necessary for post processing via digital gain using drive voltages supplied to the liquid crystal assembly at a contrast to noise ratio of 5, according to one aspect.

FIG. 17 illustrates a diagram for shortening integration times of a fluorescent image using a liquid crystal shutter assembly, according to one aspect.

FIG. 18A illustrates an image of fluorescent cubes in motion on a linear stage taken using bright background subtraction, according to one aspect.

FIG. 18B illustrates an image of fluorescent cubes in motion on a linear stage taken using a liquid crystal shutter assembly, according to one aspect.

FIG. 19 shows using a liquid crystal shutter assembly configured to visualize a peptide-fluorophore conjugate (tozuleristide).

FIGS. 20A-20B illustrate an example of front to back reflectivity ratio (FBR) (FIG. 20A) of approximately 10:1 over the visible spectrum and impact on the total reflectivity (FIG. 20B) of the beam splitter designed to be filtered by the LC shutter. If the FBR of the beam splitter designed to be filtered by the LC shutter targeted a different FBR (e.g, an FBR of approximately 20:1), the FBR curve would still vary with wavelength but have an average value of 20 across the visible spectrum.

FIG. 20A shows the FBR of the beam splitter designed to be filtered by the LC shutter in a range across the visible spectrum (i.e., between 400 nm and 700 nm wavelengths) for either the S polarized light (FBR S) or the P polarized light (FBR P) respectively. The minimum FBR of 10:1 in this example is noted.

FIG. 20B shows the effect of the polarizers on total reflectivity of the beam splitter designed to be filtered by the LC shutter in a range across the visible spectrum (i.e., between 400 nm and 700 nm wavelengths) (“total reflectivity”) as compared to the minimum reflectivity (11%) without the use of polarizing filters.

### DETAILED DESCRIPTION

Multi-spectral imaging is an important technique in regard to viewing spectral characteristics of light into a single image. The most common example of multi-spectral imaging is color vision. The human eye has three different chemistries in its rods and cones which allow the capture of light of different colors. Those three colors; red, green, and blue are then displayed as a single color image in the human mind. Digital color cameras emulate the biology of the eye by using different color filters on individual pixels of the sensor. Dedicating different pixels for different colors effectively trades spatial resolution for spectral information. The advantages of adding information into images using color bands can be applied in imaging technology. In addition, the power of multi-spectral imaging can be extended into wavelengths of light that are not directly visible to the eye. As the range of spectral information is broadened, an increasing amount of information can be condensed into a visible image. An interesting application of multi spectral imaging is that of mixing visible (VIS) and near infrared (NIR) images. This combination is empowered by CCD (charge-coupled device) and CMOS (complementary metal-oxide semiconductor) silicon cameras having good sensitivity in both the visible and NIR range. Mixing these two spectral bands can be done with multiple cameras or using, for example, a single camera that has dedicated NIR pixel filters on the sensor. Moreover, in some embodiments, one or more sensors that detect these varied spectral bands are present in the system. Alternatively, temporal multiplexing allows a single image sensor to take full resolution images in each spectral band but at the cost of time since each image must be taken in series. Filter wheels or motorized mirrors are traditionally used for switching filters but are typically too slow for optimal video rate switching speeds necessary for real time imaging. Temporal multiplexing may also be applied to multiple imaging sensors in a system (e.g., a multiple camera or multiple sensor system) but the same limitations to cost of time and video rate apply.

Multiplexing spectral bands are also used for fluorescence imaging. Fluorescence is an energy transfer mechanism where light of one color (wavelength) is absorbed and then released as a different color (wavelength). The emitted photons are typically of lower energy and thus a longer wavelength than that of the absorbed photons (Stokes's shift). Fluorescence is often observed by the human eye when the excitation wavelength is in the UV and thus not perceived by the human eye, but then emits in the visible. Fluorescence is also observed by instruments and sensors wherein that the excitation wavelength is in any spectrum not perceived by the instrument or sensor, but then emits a wavelength in the range of the instrument or sensor designed to receive the detectable fluorescent emission signal.

A fluorophore is a molecule which absorbs and then emits the light and so the specific fluorophore is responsible for both the excitation and emission spectral bands. One family of fluorophores, for example, including cyanine dyes and the Indocyanine Green dye family (ICG), both absorb and emit light in the near infrared spectrum. These NIR fluorophores are of particular importance when imaging biological samples due in part to their absorption and emission spectra both residing near 830 nm which is considered to be a primary “biological window” for transmission in tissue. ICG is also useful in medical and diagnostic applications. Combining a fluorescence image with a visible spectrum image can be an insightful tool for such biological applications. Infrared and near infrared are particularly beneficial wavelength ranges for medical imaging, but other fluorescent dyes and moieties may be used in detection systems using liquid crystal shutter assemblies described herein.

Furthermore, fluorescence imaging may be captured in real time or near real time using video image processing to detect changes over time. In numerous instances, video rate switching of NIR fluorescence with visible wavelengths may require multiple sensors or cameras, while in some instances a single camera system may be employed. Moreover, when used during NIR fluorescence, the excitation for the fluorophore can be turned on and off to control known temporal states which allows for background subtraction methods. Liquid crystal (“LC”) shutters are an assembly comprised of two linearly polarized filters with a liquid crystal media in between the two filters. The polarizing filters elements have the ability to block light of a polarization different than the filter orientation. The liquid crystal material does not block light but has the ability to rotate the polarization of light which passes through the material. The orientation of the crystals in the LC material will change the extent that the polarization of the light is rotated and thus change if the polarization of the passing light is rotated or not. The combination of two filters and an LC media along with an applied electric field can be assembled to create a shutter that will block or transmit light depending on the applied electric field. When the polarization orientation of the light which passed through the LC material is aligned with the exit polarizer, the light will pass with minimal attenuation. This is considered an open state of the shutter. When the electric field is adjusted to change the polarization orientation of light such that it is rotated at 90° from the exit polarizer, the light will be blocked. This is considered the closed state of the LC shutter assembly.

Due to the spectral bandwidth limitations of the polarizing elements incorporated into a liquid crystal shutter assembly, light that is out of band (e.g., outside of the spectral bandwidth limitations, also referred to as out of band light) of the high extinction ratio range of the polarizing elements is conventionally blocked or diverted by other spectral elements (filters) prior to being incident on the first polarizing element. By using these additional spectral filters, the extinction ratio of the liquid crystal shutter is assured.

In the methods and systems of the disclosure, rather than diverting the out of band incident light away from the liquid crystal assembly which would typically occur, the out of band light is intentionally allowed to be incident on the polarizing element and thus the liquid crystal shutter assembly. The light that is out of band will not be affected by the polarizers or the orientation of the liquid crystal media and thus pass through the shutter. Designing the optical system this way would traditionally yield a poor or minimal extinction ratio and thus defeat the function of the liquid crystal shutter assembly. In the methods and systems of the present disclosure, the spectral characteristics of the polarizing elements are chosen such that the light leaked through the shutter assembly is engineered to align to the NIR emission of the fluorophore and thus the leaked light is both intentional and beneficial. This configuration is counter to traditional good practices for liquid crystal shutter applications. Liquid crystal shutters are used to attenuate light in a controlled manner by adjusting the level of an applied electric field. A liquid crystal shutter used in this way has limited effective spectral range most commonly limited by the polarizing technology of the polarizing filters. According to various aspects, the liquid crystal shutter assemblies of the present disclosure take advantage of the various wavelength ranges that fail to become polarized by the polarizing filters. Alternatively, polarizing beam splitters can be used in combination with or, alternatively, instead of polarizing filters.

Wavelengths of light which are unsuccessfully polarized by the linear polarizing filters are typically considered to be outside the working range of the liquid crystal shutter assembly. Rather than work within the typical working range for liquid crystal shutter assemblies, the liquid crystal shutter assemblies of the present disclosure take advantage of the various wavelength ranges that are outside of the effective range of the linear polarizers and thus take advantage of spectral ranges previously considered to be detrimental in typical liquid crystal applications.

The terms “liquid crystal”, “LC”, “LC cell”, “LC media”, “LC material” “liquid crystal cell”, “liquid crystal material” or “liquid crystal media” and the like can be used interchangeably herein to refer to a liquid crystal material, media, or cell which is non-limiting, for example, including material, media, or cells comprising thermotropic liquid crystals, and lyotropic liquid crystals, Pi cell, Twisted Nematic (“TN”) cell, liquid crystal displays (LCDs), liquid crystals with photoconductor properties, or liquid crystals receptive to development and tuning under an electric field. It is understood that liquid crystals can occur in a state between the crystalline (solid) and isotropic (liquid) states. There are many types of liquid crystal states, depending upon the amount of order in the liquid crystal material.

The terms “liquid crystal shutter”, “liquid crystal assembly”, “liquid crystal shutter assembly” “LC shutter”, “LC assembly”, or “LC shutter assembly” can be used interchangeably herein to refer to a liquid crystal associated with, used in conjunction with, together with, including, or harboring one or more polarizers. Exemplary LC shutters are described herein.

The terms “polarize”, “polarizer”, or “polarizing” particularly when used when describing a component or element are understood to describe a variety of components or elements that can be functionally exchanged and used for methods that transmit light of a specific polarization orientation. For example, the LC assemblies described herein can use a variety of polarizing components elements, including but not limited to one or more polarizing beam splitters, polarizing filters, alone or in combination with other polarizing components or elements.

The terms “excitation energy” and “excitation light” are used interchangeably in this disclosure and mean to provide an excitation light to a fluorophore, or to produce an emission light from the fluorophore. Excitation energy or excitation light is also used to illuminate a fluorophore or other substance or molecule and cause an emission light.

The terms “video rate”, “video rate imaging”, “video frame rate”, “frame rate”, or “real-time” and “in real time” in the context of images, video, frame rates and video rates, particularly when used when describing speed, visualization and clarity of an image or imaging system is the number of unique or different frames or images in time used. Video rate is often measured in frames per second (“fps”), which defines how many frames or images there would be in the video in any given second. Video rate can also be measured in bitrate standard. A real time frame rate can be approximately 25 fps, or between 30 and 50 fps. Real time video frame rates can also range between 16 to 120 fps, inclusive. Video rate is a measure which affects a system's responsiveness to motion. For example, the higher the video rate the sharper the image will appear on moving images. A higher frame rate refers to more images being displayed in a second, resulting in smoother and sharper video quality and potentially giving a sharper edge to the image.

The terms “video rate sampling duration” and “exposure time” may be used interchangeably in this disclosure. The video rate sampling duration or exposure time may be the duration an imaging sensor or camera images a sample. Video rate sampling duration can also be described with respect to LC shutters and LC assemblies described herein, for example, in describing the rate of toggling between open and closed states (i.e., period between open and closed states) or two voltage sources, or drive voltage source toggles in the system. The voltage supplied to the system may toggle the LC shutter between the open state and the closed state at a video rate sampling duration ranging between 1 ms and 10 ms, between 2 ms and 20 ms, or approximately 1 ms, 2 ms, 3 ms, 4 ms, 5 ms, 6 ms, 7 ms, 8 ms, 9 ms, 10 ms, 11 ms, 12 ms, 13 ms, 14 ms, 15 ms, 16 ms, 17 ms, 18 ms, 19 ms, 20 ms between the open and closed states. Shorter time between toggling in open and closed states in LC shutters and LC assemblies described herein allows for a shorter video rate sampling duration, effectively increasing video rate resulting in more images being displayed in a second, therefore resulting in smoother and sharper video quality. Higher video rate, from a shorter video rate sampling duration, also results in improved video quality, reduces motion blur, sharper images, and images with clear boundaries and reduces artifacts.

The liquid crystal shutter assemblies and methods disclosed herein may be used in a variety of imaging systems and methods. By way of example and not limitation, the liquid crystal shutter assemblies of the present disclosure may be incorporated into one or more systems and methods for fluorescence imaging disclosed in: PCT/US2014035203 entitled “Systems and methods for recording simultaneously visible light image and infrared light image from fluorophores,” published as WO 2014/176375 on Oct. 30, 2014; PCT/US2019/024689 entitled “Systems and methods for simultaneous near-infrared light and visible light imaging,” published as WO 2019/191497 on Oct. 3, 2019; PCT/US2020/053746 entitled “Systems and methods for vascular and structural imaging,” published as WO 2021/067563 on Apr. 8, 2021; and PCT/US2021/039177 entitled “Systems and methods for simultaneous near-infrared light and visible light imaging,” published as WO 2021/263159 on Dec. 30, 2021, (collectively referred to as the “Blaze Bioscience systems”), each of which is incorporated herein by reference in its entirety. It is also understood that the liquid crystal shutter assemblies of the present disclosure may be incorporated into any one or more systems and methods for fluorescence imaging known in the art.

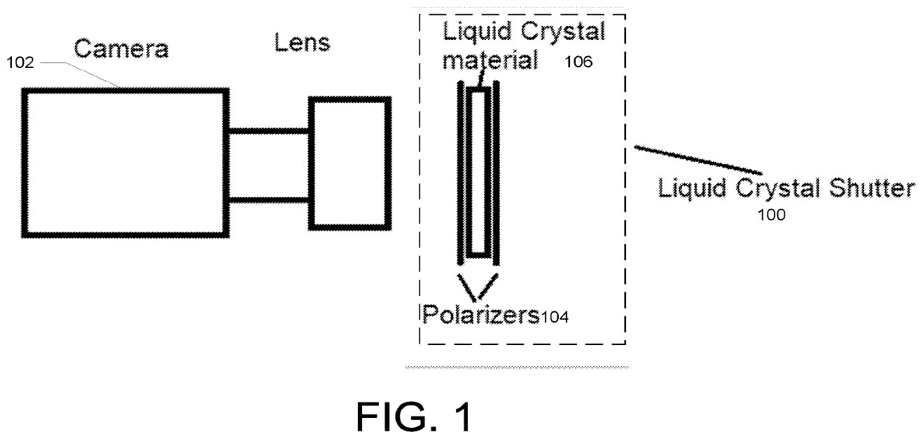

In one aspect of the present disclosure, it is desirable to use a liquid crystal shutter assembly 100 in conjunction with at least one sensor or camera 102 with sufficient sensitivity in the spectral range of the visible and NIR. As shown in FIG. 1, the LC shutter assembly 100 further includes polarizers 104 and a liquid crystal media 106 within the LC shutter assembly 100.

According to one embodiment, two polarizers 104 are integrated within the LC shutter 100, as shown in FIG. 1. In other embodiments, one or more polarizers 104 may be separate components that are positioned in the same optical path and aligned on either side of a LC media cell 106. In yet other embodiments, multiple LC media cells 106 each associated with one or more polarizers 104 may be used to form a LC assembly 100. Stacking polarizers and liquid crystal media this way is used to increase the extinction ratio compared to a single cell LC shutter assembly. Alternatively, one or more LC assembly 100 comprising one or more LC cells 106 and one or more polarizers 104 may be used in conjunction with any combination of additional LC cells 106 and/or polarizers 104.

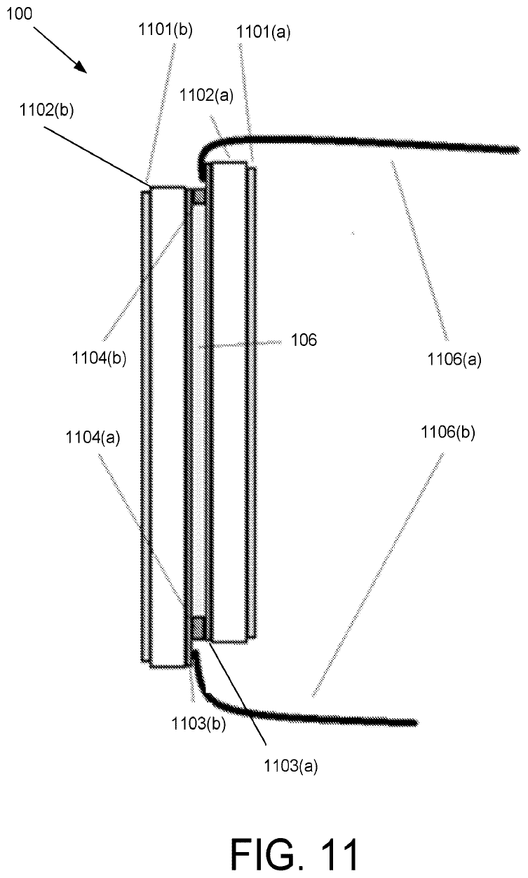

According to one embodiment, the two polarizers 104 may each comprise a substrate layer 1102(a), 1102(b), as shown in FIG. 11. The first substrate layer 1102(a) of the first polarizer and the second substrate layer 1102(b) of the second polarizer may be glass substrate layers. The glass substrate layers may be configured to allow light to pass through. A first polarizing film 1101(a) may be adhered to the first substrate layer 1102(a) and a second polarizing film 1101(b) may be adhered to the second substrate layer 1102(b), as shown in FIG. 11. The first polarizing film 1101(a) and the second polarizing film 1101(b) may be cross-polarized (e.g., of opposite orientations).

In an aspect, the first substrate layer 1101(a) may have an electrically conductive layer 1103(a) located on a side of the substrate layer 1102(a) opposite the first polarizing film 1101(a) and adjacent to the LC media 106. The second substrate layer 1102(b) may have an electrically conductive layer 1103(b) located on a side of the substrate layer 1102(b) opposite the second polarizing film 1101(b) and adjacent to the LC media 106.

In an aspect, the first electrically conductive layer 1103(a) and the second electrically conductive layer 1103(b) may both comprise indium tin oxide (“ITO”). As used herein, it is understood that transparent conducting films or layers comprise any thin films of optically transparent and electrically conductive material. In other examples the first electrically conductive layer 1103(a) may comprise any transparent conductive film or layer including those comprising indium tin oxide (“ITO”), low- or high resistance transparent conductive film, transparent conducting oxide (“TCO”), indium zinc oxide (“IZO”), other tin oxide (Sno2) based films including indium-free un-doped tin dioxide, Cu and Ga-doped zinc oxide (ZnO) films (“GZO”), aluminum zinc oxide (“AZO”), and cadmium stannate (“CTO”). The second electrically conductive layer 1103(b) may comprise any transparent conductive film or layer including those comprising indium tin oxide (“ITO”), low- or high resistance transparent conductive film, transparent conducting oxide (“TCO”), indium zinc oxide (“IZO”), other tin oxide (Sno2) based films including indium-free un-doped tin dioxide, Cu and Ga-doped zinc oxide (ZnO) films (“GZO”), aluminum zinc oxide (“AZO”), and cadmium stannate (“CTO”). In some examples, the first electrically conductive layer 1103(a) and the second electrically conductive layer 1103(b) may comprise the same material. In other examples, the first electrically conductive layer 1103(a) and the second electrically conductive layer 1103(b) may comprise different materials. The first electrically conductive layer 1103(a) may be soldered to the first substrate layer 1102(a). The second electrically conductive layer 1103(b) may be soldered to the second substrate layer 1102(b).

In another aspect, the first electrically conductive layer 1103(a) and the second electrically conductive layer 1103(b) may be kept thin to maintain a high transmission of NIR light through the LC shutter 100. In some embodiments the electrically conductive layer (whether the first layer or second layer) can be 10-50 nm think inclusive, less than 25 nm thick, less than 50 nm thick, between 50 nm and 250 nm thick, between 75 nm and 165 nm thick, or over 250 nm thick, depending on the materials used for the transparent conductive film or layer. In some aspects, the transparent conductive film or layer is 10-30 nm thick, 30-50 nm thick, 50-70 nm thick, 70-90 nm thick, 90-110 nm thick, 110-130 nm thick, 130-150 nm thick, 150-170 nm thick, 170-190 nm thick, 190-210 nm thick, 210-230 nm thick, or 230-250 nm thick. In some aspects, the transparent conductive film or layer is 20 nm thick (+/−5 nm), 30 nm thick (+/−5 nm), 40 nm thick (+/−5 nm), 50 nm thick (+/−5 nm), 60 nm thick (+/−5 nm), 70 nm thick (+/−5 nm), 80 nm thick (+/−5 nm), 90 nm thick (+/−5 nm), 100 nm thick (+/−5 nm), 110 nm thick (+/−5 nm), 120 nm thick (+/−5 nm), 130 nm thick (+/−5 nm), 140 nm thick (+/−5 nm), 150 nm thick (+/−5 nm), 160 nm thick (+/−5 nm), or 170 nm thick (+/−5 nm). In some embodiments the electrically conductive film or layer may be heated or nonheated.

In another aspect, the LC media 106 may be located between the first electrically conductive layer 1103(a) and the second electrically conductive layer 1103(b), as shown in FIG. 11. The LC media 106 may be sealed within the LC shutter 100 by the first electrically conductive layer 1103(a), the second electrically conductive layer 1103(b), a first spacer 1104 (a) and a second spacer 1104 (b). The LC shutter 100 may include a first electrical connector 1106 (a) and a second electrical connector 1106 (b). The first electrical connector 1106 (a) may be connected to the first electrically conductive layer 1103(a). The second electrical connector 1106 (b) may be connected to the second electrically conductive layer 1103(b). The first electrical connector 1106 (a) may be configured to supply a voltage from the drive voltage source to the first electrically conductive layer 1103(a). The second electrical connector 1106 (b) may be configured to supply a voltage from the drive voltage source to the second electrically conductive layer 1103(b). When a voltage is supplied to the first electrically conductive layer 1103(a) and the second electrically conductive layer 1103(b), an electric field may be applied to the LC media 106. The voltage supplied by the drive voltage source to the first electrically conductive layer 1103(a) and the second electrically conductive layer 1103(b) may be a bi-polar square wave.

When no electric field (e.g., no voltage) is applied to the LC media 106, the LC media 106 may rotate the polarization of light passing through it. When the first polarizing film 1101(a) and the second polarizing film 1101(b) are cross polarized (e.g., of opposite orientations), this state of the LC shutter 100 is called the open state. By rotating the polarization of the light, the light may pass through the second polarizing film 1101(b) since the light will have the same polarization as the second polarizing film 1101(b) (e.g., the light is polarized by the first polarizing film 1101(a) then rotated by the LC media 106 to the same polarization of the second polarizing film 1101(b)). In this example, the two polarizing films may appear to be aligned to each other. The LC shutter 100 may appear transparent (e.g., no attenuation of light) when no electric field is supplied to the LC media 106. When an electric field is applied to the LC media 106, the LC media 106 does not rotate the light passing through it. When the LC media 106 does not rotate the light, the light has the polarization of the first polarizing film 1011 (a) when it reaches the second polarizing film 1101(b) and therefore is blocked by the cross oriented second polarizing film 1101(b). This state of the LC shutter 100 is the closed state. The voltage supplied may toggle the LC shutter between the open state and the closed state.

In another aspect, the first polarizing film 1101(a) and the second polarizing film 1101(b) may have the same polarization (e.g., same orientation). In this example, when no electric field (e.g., no voltage) is supplied the LC media 106 may rotate the polarization of light passing through it. In this example, by rotating the polarization of light, the light may be blocked by the second polarizing film 1101(b) because the rotated polarization of the light has an opposite polarization to the second polarizing film 1101(b). In this example, when an electric field is supplied to the LC media 106, the LC media 106 does not rotate the light. In this example, when the LC media 106 does not rotate the light, the light has the polarization of the first polarizing film 1101(a), and therefore the polarization of the second polarizing film 1101(b) allowing the light to pass through the second polarizing film 1101(b). In this example, the effect of the voltage supply can be reversed (e.g., no voltage provides the closed state and supplying a voltage provides the open state) by changing the orientation of the polarizing films 1101(a), 1101(b).

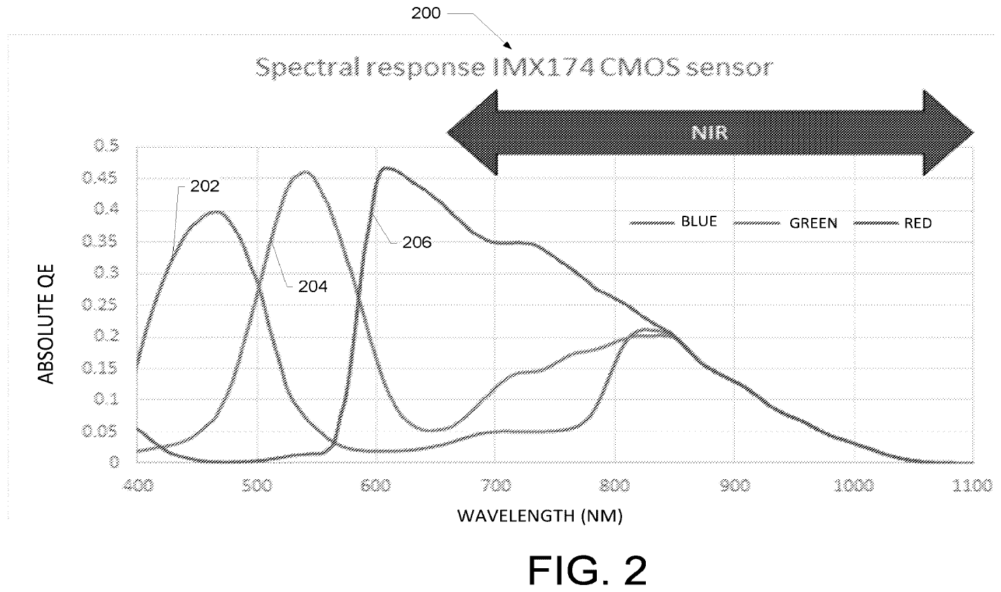

In various aspects, the camera 102 incorporates one or more color filters that are highly transmissive in the NIR spectrum. As shown in FIG. 2 which illustrates the spectral response 200 of an exemplary camera, the camera 102 may include blue, green, and red filters on the sensor pixels, generally indicated by 202, 204, and 206 respectively, to isolate incoming light in the aforementioned wavelengths. According to various aspects, the camera 102 is a multispectral camera. In other aspects, the camera is configured for hyperspectral imaging. In various aspects, the polarizer 104 and the LC cell 106 effectively act as a long-pass filter, where the shorter wavelengths of light in the visible spectrum can be blocked or allowed to pass based on the applied electric field, thereby controlling the light received at the camera 102. In various aspects, unattenuated visible light and an emission light may be directed to the LC shutter 100.

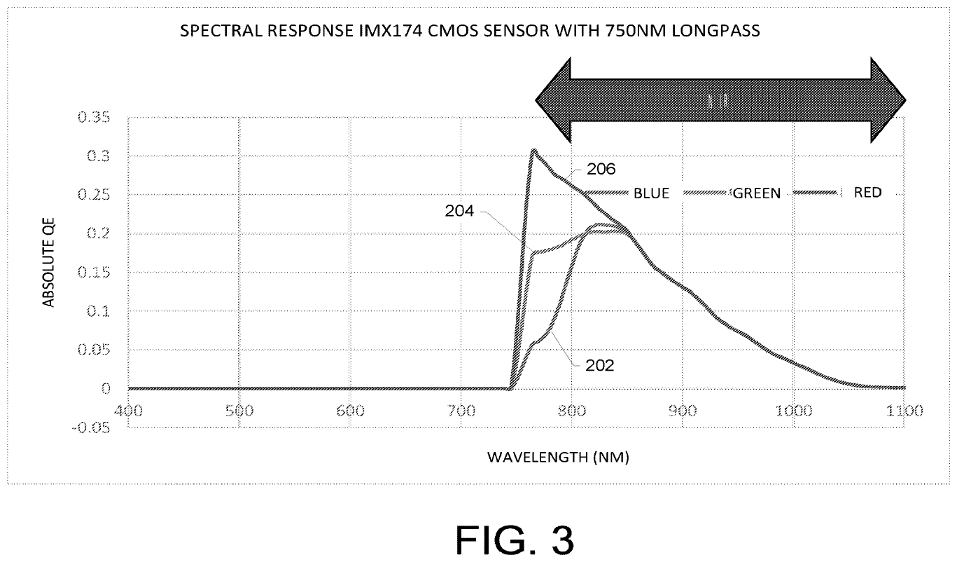

As shown in FIG. 3, multispectral imaging systems typically include an NIR long-pass filter which is used to separate out the visible light and the NIR light. The long-pass filter allows only the NIR light from the light source to pass through to an imaging sensor, such as the camera 102. In various aspects of multispectral imaging, the use of the NIR long-pass filter is desirable in that all three color pixels 202-206 depicted act more like a monochrome camera, which carries the full resolution of the sensor. In order to time multiplex a visible and NIR image, the long-pass filter is typically moved in and out of the image path. In practice, moving a physical filter in an out of the image path can be accomplished using a spinning filter wheel or rotary solenoid, however these methods are difficult to do precisely at video speed, and are prone to synchronization error, audible and sub-audible noise, thermal noise, and vibrations (i.e., any of the foregoing commonly referred to as noise, or specifically as the context requires), and mechanical failure. Consequently, the liquid crystal shutter assembly of the present disclosure can act as a long-pass filter used to address these limitations in order to obtain images at video speed, and reduce or eliminate the aforementioned issues, including reducing or eliminating synchronization error, noise, and mechanical failure. According to various aspects of the present disclosure, the reduced extinction ratio at long wavelengths effectively converts the liquid crystal assembly into a long-pass filter with variable attenuation in the short wavelength light which is within the effective range of the polarizers. In many instances, linearly polarized filters suffer from undesirable disadvantages. The first disadvantage is that at least 50% of the unpolarized light is lost to absorption or reflection such that only polarized light remains. In the case of a liquid crystal assembly using such polarizers, this light is lost from the first polarizer. Moreover, in low light applications, such as found during NIR fluorescence imaging, signal attenuation by approximately 50% or more can lower the signal to noise (SNR) ratio to such an extent that increased exposure times are necessary to capture useful data. Consequently, the need for longer exposure times is often detrimental for capturing data at video rates.

The second shortcoming of using these types of polarizers is that they are not effective for all wavelengths of incoming light. As readily understood, a polarizer becomes effectively transparent outside of its range of polarization. In other words, a polarizer may be ineffective to polarize light outside of its wavelength range of polarization. Thus, at particular wavelengths, the polarizer fails to polarize the incoming light.

Counter to prevailing thought, according to various aspects, the system and methods of the present disclosure take advantage of these previously undesirable features or disadvantages. The LC shutter assembly 100 is able to operate in three states. These include “State 1” also referred to as the “open state”, where the unpolarized light that is within the functional range of the linear polarizers becomes partially attenuated by the first linear polarizer but is generally allowed to pass through the second polarizer based on the polarization rotation of the liquid crystal. The open state can be used to image visible light from a sample (e.g., reflected visible light). “State 2” also referred to as the “closed state” is where unpolarized light is partially attenuated by the first linear polarizer, and then almost entirely blocked or attenuated by the second linear polarizer due to an alternate rotation of the liquid crystal phase state. The ability to attenuate light in this closed state relies on the effectiveness of the linear polarizers to fully polarize the light. In various aspects, the characteristics of the polarizing filters allow an LC shutter to achieve two to four orders of magnitude in extinction ratio within the spectral range of the polarizing filters when comparing the Open and Closed states. In an exemplary embodiment of visible and NIR spectral imaging, the polarizing filters in the system and methods of the present disclosure are chosen to be effective in the visible spectrum and thus the open and closed states only affect visible light. Alternatively, polarizing beam splitters can be used instead of polarizing filters.

Lastly, “State 3” refers to a state where the light passing through an LC shutter assembly, such as, but not limited to the LC assembly 100, has spectral characteristics outside of the polarizers effective working range and thus passes through the shutter assembly with very little attenuation. In the State 3 spectral range, the unpolarized light fails to become polarized or attenuated and thus the LC phase state is inconsequential. In this spectral range, over 90% transmission can be achieved with proper anti reflection coatings on the surfaces of the polarizing filters. State 3 which is traditionally considered a failed state due to a low extinction ratio becomes a high transmission state regardless of the liquid crystal phase state.

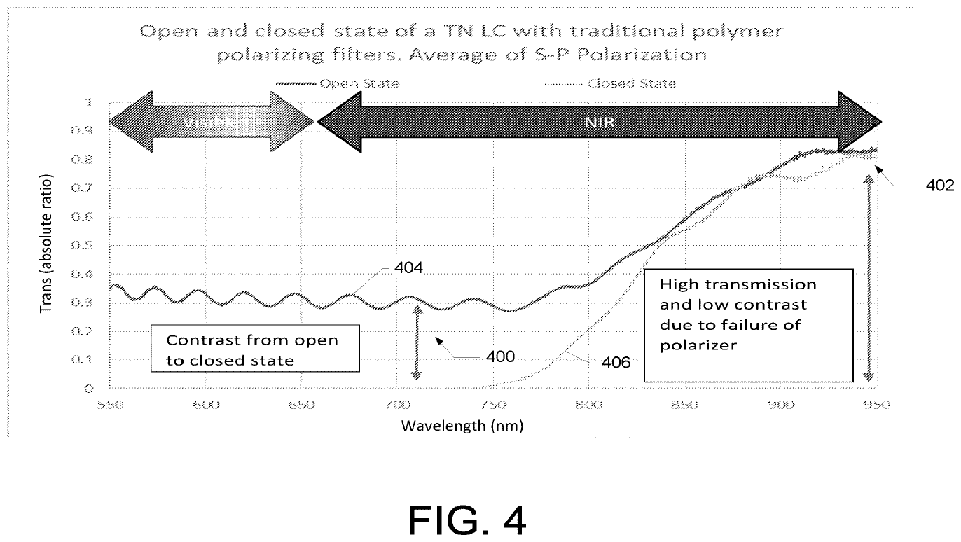

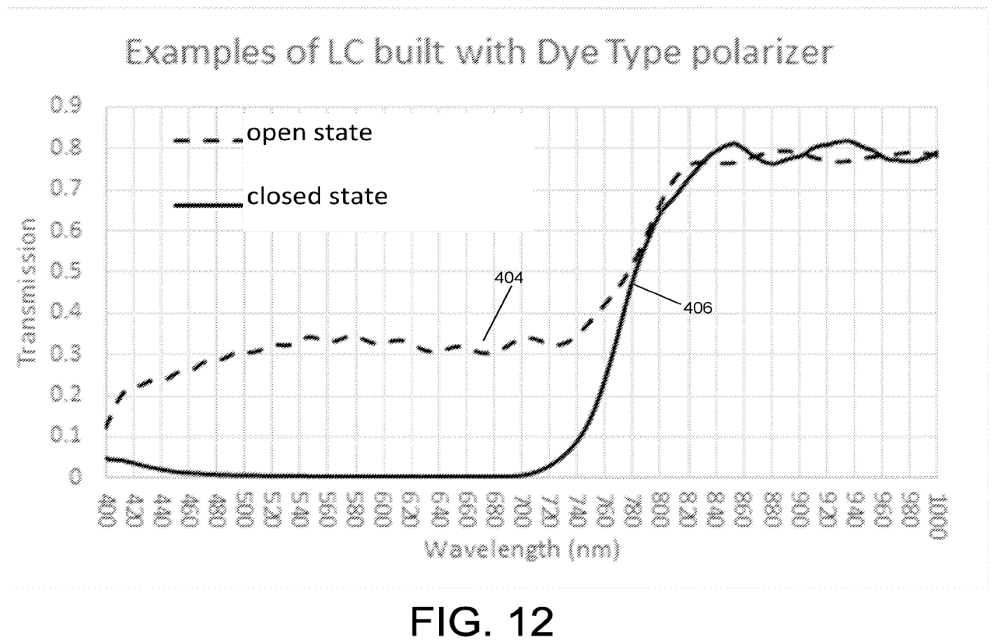

According to one aspect, the LC shutter assembly 100 uses both the incoming light polarized by the polarizer 104 (State 1), as well as incident light that fails to be polarized by the filter and reaches the LC cell 106 with little attenuation for filtering through the LC shutter 100 in an open or closed state (States 1 and 2). As shown in FIG. 4, the extinction ratio of light, indicated as 400, entering the LC shutter 100 between the open state and the closed state of the shutter decreases as the polarizer fails to attenuate the light in the NIR spectrum. As shown in FIG. 12, when the LC shutter 100 is in the open state 404 (State 1), light in the visible spectrum (e.g., having wavelengths between 400 nm and 700 nm) may be transmitted through the LC shutter 100. When the LC shutter 100 is in the closed state 406 (State 2), light in the visible spectrum (e.g., having wavelengths between 400 nm and 700 nm) may not be transmitted through the LC shutter 100. The same general method will work on other optical systems that have light sources with similar spectral characteristics. On systems which have different spectral contributions to their light source, other polarizers may be chosen to yield the appropriate blocking/attenuation of light.

As shown, attenuation caused by the polarizer 104 fails at the longer wavelengths. The LC cell 106 no longer receives about 50% of the incoming (unpolarized) light by virtue of the failed 50% attenuation at those longer wavelengths. In some examples, the failed attenuation is a result of a specifically engineered light-cut off range for viewing of a desired light (e.g., fluorescence). As such, the contrast from the open state 404 to closed state 406, generally indicated as 402, also decreases.

According to various aspects of the present disclosure, this contrast decrease becomes a beneficial feature during State 3 operation, when superimposing a visible light image over a NIR fluorescence image. The resulting superimposed composite image(s) and/or video(s) provide greater clarity, details, and information to medical personnel regarding presence or absence of disease states (e.g., including presence of tumors, cancer, vascular malformations, inflammation or other), variations in tissue and sub-tissue structure, anatomical structures of interest, and surrounding tissue or samples of the foregoing, whether examined in vivo or ex vivo.

In yet another aspect, the amount of visible light can be adjusted by operating the LC phase in between State 1 (the open state) and State 2 (the closed state). These in between states are practically controlled by adjusting the applied voltage and thus the electric field of the LC cell and allow for variable attenuation of the light without affecting the light in the failed state (State 3).

According to various aspects, the LC shutter assemblies, such as the LC shutter assembly 100, of the present disclosure may be used in various states and further may be operated when in between various open and closed states to enable selection of desired wavelengths in the failed state depending on the application of the systems or methods of the present disclosure. In another aspect, one “in-between” state of operation has at least 50% attenuation and up to a maximum attenuation of 100% based on the extinction ratio of the filters. Operating in between the open and closed states allows the adjustment or customization of the ratio of visible light to NIR light. The NIR signal is typically at much lower intensities than the visible light present in a system and as such would typically require different gain settings or exposure times in the image sensor. By adjusting the amount of visible light attenuation between the open and closed states the visible light levels can be scaled to match that of the weak NIR fluorescence signal and thus allow for both spectral ranges to use the same camera settings.

In another aspect, the video rate sampling duration (i.e., exposure time) may be different for imaging a visible light image and an emission light from a fluorophore (e.g., fluorescence or fluorescent image). The liquid crystal shutter assembly may be placed in the open state for imaging a visible light image. The liquid crystal shutter assembly may remain in the open state for about 2 ms to about 6 ms for a video rate sampling duration to image the visible light image. The liquid crystal shutter assembly may be placed in the closed state to image emission light from a fluorophore. The liquid crystal shutter assembly may remain in the closed state for about 8 ms to about 16 ms for a video rate sampling duration to image the emission light from the fluorophore. In other examples, the video rate sampling duration for a visible light image may be about 1 ms, about 2 ms, about 3 ms, about 4 ms, about 5 ms, about 6 ms, about 7 ms, about 8 ms, about 9 ms, about 10 ms, about 11 ms, about 12 ms, about 13 ms, about 14 ms, about 15 ms, about 16 ms, or higher. In another example, the video rate sampling duration to image the emission light from the fluorophore may be about 5 ms, about 6 ms, about 7 ms, about 8 ms, about 9 ms, about 10 ms, about 11 ms, about 12 ms, about 13 ms, about 14 ms, about 15 ms, about 16 ms, about 17 ms, about 18 ms, about 19 ms, about 20 ms, about 25 ms, about 30 ms, or higher.

### Engineered Polarizer

In order to take full advantage of the failed polarizer state (i.e. when the incoming wavelengths fall outside the polarizing range of the polarizer), the polarizer 104 may be specifically manufactured or engineered to cut-off at a specific desired wavelength (e.g., light cut-off). For example, when performing imaging fluorescence with an indocyanine Green (ICG) dye, the spectral emission is in the range of 750 nm-950 nm, or in the range of 800 nm-880 nm, and peaks at around 830 nm. For example, in some embodiments, the polarizer 104 for the liquid crystal shutter assembly 100 is chosen to lose effectiveness between 650 and 800 nm, or 600 nm and about 850 nm, or between about 700 nm to about 850 nm, about 800 nm to about 950 nm, about 800 nm to about 880 nm, about 775 nm to about 795 nm, or about 785 nm. In additional embodiments, the polarizer 104 (or two polarizers) are configured to lose effectiveness or have a light cut-off range of about 600 nm to about 620 nm, about 620 nm to about 640 nm, about 640 nm to about 660 nm, about 660 nm to about 680 nm, about 680 nm to about 700 nm, about 700 nm to about 720 nm, about 720 nm to about 740 nm, about 740 nm to about 760 nm, about 760 nm to about 780 nm, about 780 nm to about 800 nm, about 800 nm to about 820 nm, about 820 nm to about 840 nm, about 840 nm to about 860 nm, about 860 nm to about 880 nm, or about 880 nm to about 900 nm. In other embodiments, the polarizer 104 (or two polarizers) may be configured to lose effectiveness or have a light cut-off range below about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, or about 450 nm to about 500 nm. In other embodiments, the liquid crystal shutter assembly is chosen to lose effectiveness by 800 nm.

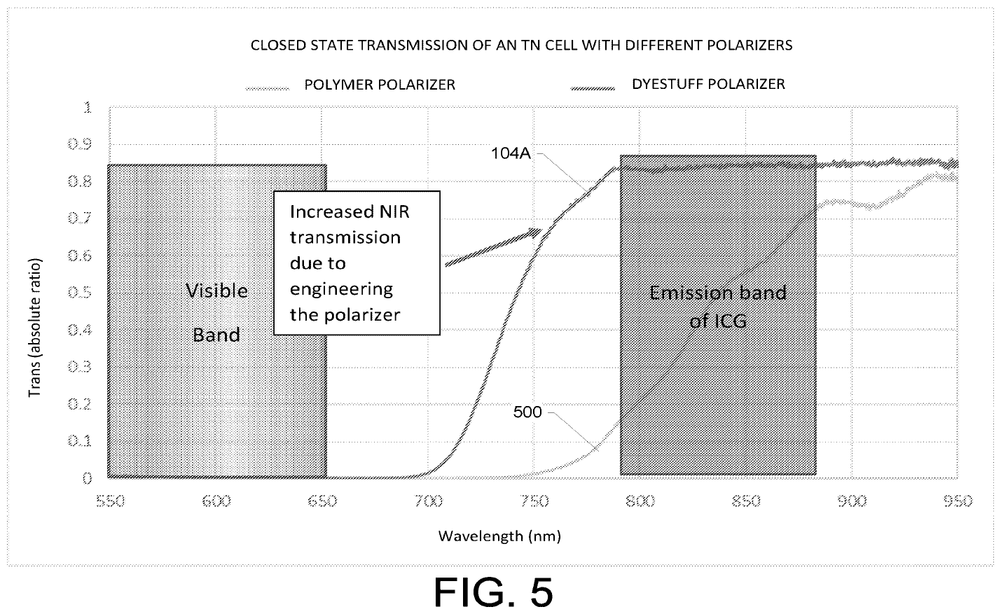

According to one aspect of the LC shutter assembly 100, approximately 50% of the fluorescence emission may be lost due to the polarizers 500, as shown in FIG. 5. In contrast, a specifically configured or engineered polarizer 104A, may be used in accordance with various embodiments. The engineered polarizer 104A is specifically chosen and/or manufactured such that the cut off wavelength for the polarizer may be tuned or engineered to a specific wavelength. For example, if ICG is the detectable fluorophore of interest the polarizer could be tuned to cut off at a desired wavelength in a range between 790 to 900 nm. In another example, another ICG fluorophore or any other fluorophores suitable for fluorescence imaging in the near-infrared range (˜ 780 nm to 2500 nm) may be used in conjunction with the polarizers 104 engineered accordingly to a specific desired cut-off wavelength. FIG. 5 illustrates different NIR transmission levels between an engineered polarizer 104A and a polymer polarizer 500 in conjunction with a closed state LC shutter.

By way of example and not limitation, polarizer 104 may be a dyestuff polarizer which is very effective in the visible range but quickly drops off in the NIR wavelength range. According to various aspects, a dyestuff polarizer is preferred as they possess high damage thresholds which are suitable to absorb large amounts of light in the visible spectrum.

In an aspect, the liquid crystal shutter assembly provides minimal attenuation of light outside of the light cut-off range of the polarizer in State 3. In some examples, light outside of the light cut-off range may be near-infrared light (NIR) or infrared light (IR). In other examples, light outside of the light cut-off range may be light with wavelengths above the visible light spectrum, wavelengths within visible light spectrum, or light with wavelengths below the visible light spectrum. Minimal attenuation provides for optimal imaging of fluorescence from a fluorophore by providing a maximum intensity of emission light to the imaging sensor and/or camera. In some examples, the emission light from the fluorophore may have a 0% to 5% attenuation when passing through the liquid crystal shutter assembly 100. In other examples, the emission light from the fluorophore may have an attenuation of about 0% to about 5%, about 5% to about 10%, or about 10% to about 15% when passing through the liquid crystal shutter assembly 100.

### Liquid Crystal Cell

According to various aspects, any of a number of liquid crystal materials may be used in the LC shutter 100. By way of example, the LC shutter 100 may include a Pi cell or a Twisted Nematic (“TN”) cell. Typically, Pi LC cells have faster opening and closing times than Twisted Nematic (TN) cells. Additionally, Pi cells can cause a chromatic shift to the color hue of an image. Pi cells are commonly used in DLP projectors to provide video rate switching. DLP projectors, however, require temperature compensating voltage control to avoid chromatic fluctuations. Though effective, the use of Pi cells may not be as desirable for use in uncooled and simplified imaging systems. In one aspect of a twisted nematic liquid crystal, applying a non-zero voltage induces a backflow in the liquid crystal causing a momentary “opening” of the cell. It yet another aspect, the backflow defines a partially open transmission state.

According to other aspects of the LC shutter assembly 100, a TN cell is preferred. While a TN cell shutter is typically slower than an equivalent Pi cell, the TN cell has more desirable spectral characteristics in that its absorption is relatively neutral within the operating spectral range. Thus, for a compact, uncooled imaging system, the chromatically natural characteristics of a TN cell are more desirable. The robust spectral characteristics of a TN cell are of great benefit. The consequence of the TN cell is their slow shutter opening time. This time can be improved with temperature.

According to various aspects, the closing time of a TN cell may be increased by increasing the applied voltage of the TN cell above its normal operating voltage. In one example, a TN cell that may be used (e.g., LC media 106) in an LC shutter 100 may be configured to be open when there is no electric field applied. In this example, the TN cell shutter may achieve very fast shutter speeds as well as a high contrast extinction ratio. An additional feature of TN cells that may ordinarily be considered a disadvantage is that the TN cell will demonstrate a “backflow” during opening when driven with high voltages. In particular, the high voltage induces a TN cell backflow causing the LC shutter to briefly open, fully close, and finally open completely.

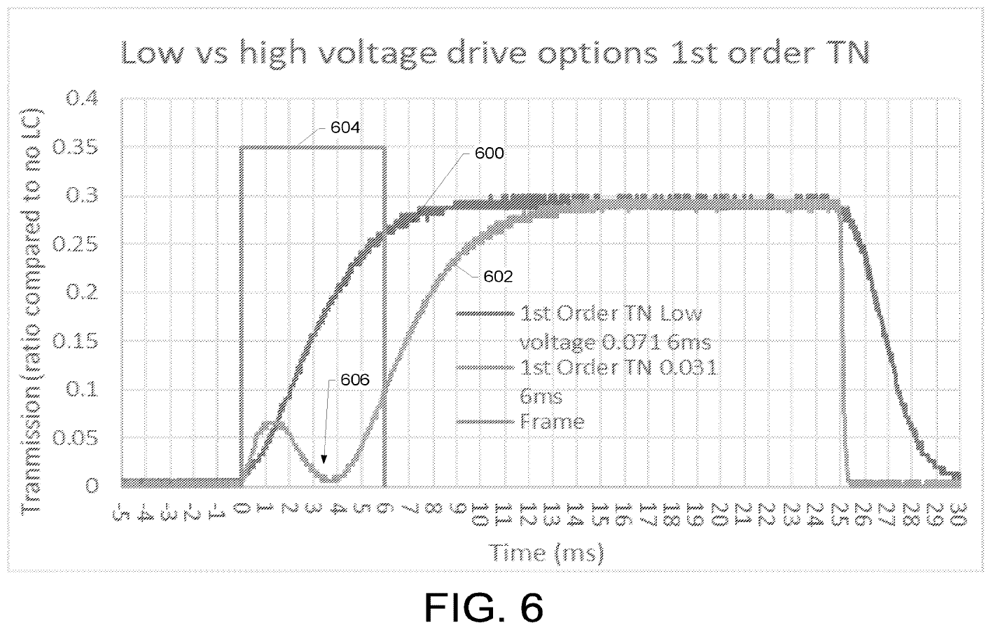

For example, FIG. 6 shows a TN cell being driven with two different voltages, a lower voltage drive 600 and a higher voltage drive 602. The lower voltage drive 600 is the one recommended by the manufacturer to avoid backflow. As shown, a 6 ms box 604 illustrates an example video rate sampling duration (i.e., exposure time) for video rate imaging. While the lower voltage drive 600 causes a more open state within the 6 ms duration, it becomes necessary to halt the voltage drive earlier to close the cell due to the slow closing time. In contrast, the higher voltage drive 602 causes a faster initial opening before the backflow closing 606 occurs. While the total transmission of light using the higher voltage drive 602 is less than that of the lower voltage drive 600 within the 6 ms window, the TN cell performance may be further modified by heating the LC cell 106.

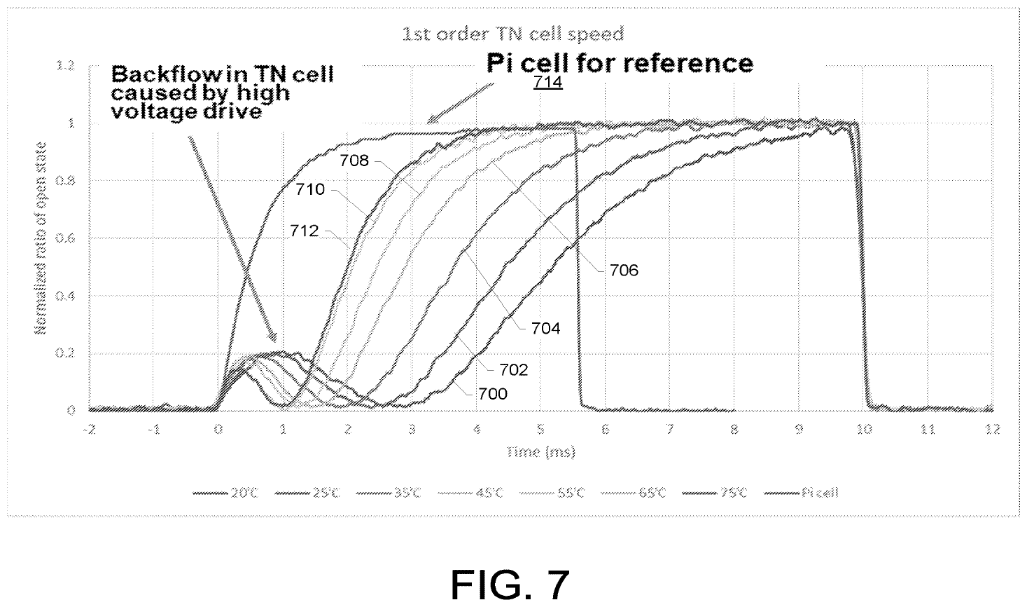

Referring to FIG. 7, the opening time of a TN LC shutter (i.e., an LC shutter configured with a TN cell as the liquid crystal cell) may be a function of temperature. In particular, opening times for a TN LC shutter at 20° C. (700), 25° C. (702), 35° C. (704), 45° C. (706), 55° C. (708), 65° C. (710), and 75° C. (712) are shown. The opening time for a Pi cell, indicated as 714 is shown for comparison. As shown, heating the LC cell 106 decreases the viscosity of the liquid crystal material in the cell. As a result, the LC shutter response speed increases thereby shortening the duration of the backflow phenomena and ultimately increasing the speed at which the LC shutter 100 fully opens. In some examples, the transition speed between the open state and the closed state is determined by the temperature of the liquid crystal and the drive voltage source. In some embodiments the LC cell 106 is heated, for example between thirty and fifty degrees Celsius.

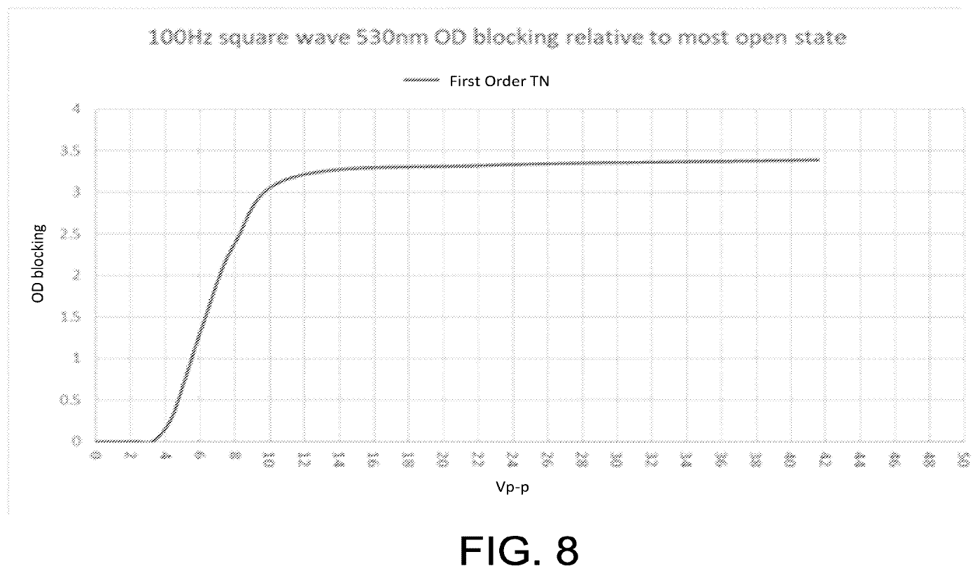

According to one aspect, the LC shutter may be heated by a dedicated heater that may be controlled to directly tune the performance of the TN cell. According to another aspect, the LC shutter 100 may be disposed inside an enclosed housing that provides an elevated temperature through waste heat generated by other components of the imaging system. As shown in FIG. 6, in response to the heated environment, the reduced backflow transmission plus the partially opened state is sufficiently high for real-time visible imaging. By installing the LC cell 106 or the entire LC shutter assembly 100 within an enclosed environment, such as but not limited to a housing of the imaging systems disclosed in aforementioned Blaze Bioscience systems, the elevated internal temperature produced by other components of the imaging systems help mitigate the backflow associated with a TN cell. By way of example and not limitation, the LC cell 106 may be held around 35-40° C. In numerous aspects, an additional benefit of driving TN cells at higher voltages is an increase in optical blocking, as shown in FIG. 8. For example, TN cells are typically driven with 0 V for the open state and some nominal low voltage for the closed state. However, a higher voltage used for fast closing time also helps increase the blocking capability of the cell. As shown, the higher peak to peak (“p-p”) drive voltage works beneficially for the application. The LC cell 106 may be driven by a DC drive voltage or an AC drive voltage.

According to one aspect, the LC cell 106 is driven at a high voltage in a range between +/−3Vp-p and +/−60Vp-p [peak to peak]. In one exemplary aspect, the LC shutter cell is driven with +/−24 V (48 Vp-p). In one exemplary aspect, the LC shutter cell is driven in a range between +/−16 V (32 Vp-p) and +/−30 V (60 Vp-p), at any value inclusive. In one exemplary aspect, the LC shutter cell is driven in a range between +/−30 V (60 Vp-p) and +/−100 V (200 Vp-p), at any value inclusive. According to various aspects, the LC shutter is driven by a square wave voltage with a 1-2 kHz period and a 50% duty cycle. In that example, the square wave voltage ranges from a minimum of 0 V for the open state to a maximum +/−24 V (48 Vp-p) for the closed state. In another aspect, the square wave drive voltage is provided as a DC drive voltage that ranges from −24 V to 24 V such that the voltage drive is approximately 48 V peak-to-peak. In another aspect, the amplitude of the bi-polar square wave has a range of +/−3 to +/−30Vp-p. In one exemplary aspect, the amplitude of the bi-polar square wave a range between +/−16 V (32 Vp-p) and +/−30 V (60 Vp-p), at any value inclusive. In one exemplary aspect, the amplitude of the bi-polar square wave ranges between +/−30 V (60 Vp-p) and +/−100 V (200 Vp-p), at any value inclusive. In another aspect, the second voltage comprises a plurality of voltages creating a bi-polar square wave voltage with an average mean value of 0 V. In this aspect, the voltage is held at the full 24 V for a closed state video rate image. In this embodiment there is no switching of the polarity of the voltage bias during the frame exposure and thus the LC shutter maintains a higher level of optical attenuation for that closed state. The voltage polarity will be flipped for the next frame which requires the closed state preserving the health and reliability of the LC shutter.

According to various aspects, the LC shutter may have a first voltage to toggle the LC shutter to the open state and a second voltage to toggle the LC shutter to the closed state. In some examples, the first voltage may be 0 V. Applying the first voltage may allow the liquid crystal shutter assembly to assume the open state. In other examples, the first voltage may be a bi-polar square wave having an amplitude in the range of +/−0.1Vp-p to +/−5Vp-p. When the liquid crystal is a Twisted Nematic liquid crystal cell, the first voltage may be 0 V. When the liquid crystal is a PiCell, the first voltage may be a bi-polar square wave having an amplitude in the range of +/−0.1Vp-p to +/−5Vp-p. In other examples, the first voltage may be a bi-polar square wave having an amplitude in the range of +/−3Vp-p and +/−60Vp-p. In other examples, the first voltage may be a bi-polar square wave having an amplitude of about +/−12Vp-p to +/−15Vp-p, about +/−15Vp-p to +/−18Vp-p, about +/−18 Vp-p to +/−21Vp-p, about +/−21Vp-p to +/−24Vp-p, about +/−24Vp-p to +/−27Vp-p, about +/−30Vp-p to +/−33Vp-p, about +/−36Vp-p to +/−39Vp-p, about +/−42Vp-p to +/−45Vp-p, about +/−45Vp-p top +/−48Vp-p, about +/−51Vp-p to +/−54Vp-p, about +/−54Vp-p to +/−57Vp-p, about +/−57Vp-p to +/−60Vp-p, or higher. The second voltage may be a bi-polar square wave having an amplitude in the range of +/−3Vp-p and +/−60Vp-p. Applying the second voltage may allow the liquid crystal shutter assembly to assume the closed state. In other examples, the second voltage may be a bi-polar square wave having an amplitude of about +/−12Vp-p to +/−15Vp-p, about +/−15Vp-p to +/−18Vp-p, about +/−18 Vp-p to +/−21Vp-p, about +/−21Vp-p to +/−24Vp-p, about +/−24Vp-p to +/−27Vp-p, about +/−30Vp-p to +/−33Vp-p, about +/−36Vp-p to +/−39Vp-p, about +/−42Vp-p to +/−45Vp-p, about +/−45Vp-p top +/−48Vp-p, about +/−51Vp-p to +/−54Vp-p, about +/−54Vp-p to +/−57Vp-p, about +/−57Vp-p to +/−60Vp-p, or higher. In further examples, the second voltage may be 0 V. In another example, the second voltage may be a bi-polar square wave having an amplitude in the range of +/−0.1Vp-p to +/−5Vp-p.

In an aspect, the bi-polar square wave may be modulated from 500 to 2000 Hz. In some examples, the drive voltage source toggles the liquid crystal between the first voltage (e.g., open state) and the second voltage (e.g., closed state) in durations from 2 ms to 40 ms inclusive, 1 millisecond to 40 milliseconds inclusive, or 2 milliseconds to 60 milliseconds inclusive, 60 milliseconds to 100 milliseconds inclusive, and up to 300 milliseconds inclusive. In other examples, the drive voltage source toggles the liquid crystal between the first voltage (e.g., open state) and second voltage (e.g., closed state) in durations from about 2 ms to about 4 ms, about 4 ms to about 6 ms, about 6 ms to about 8 ms, about 8 ms to about 10 ms, about 10 ms to about 12 ms, about 12 ms to about 14 ms, about 14 ms to about 16 ms, about 16 ms to about 18 ms, about 18 ms to about 20 ms, about 20 ms to about 22 ms, about 22 ms to about 24 ms, about 24 ms to about 26 ms, about 26 ms to about 28 ms, about 28 ms to about 30 ms, about 30 ms to about 32 ms, about 32 ms to about 34 ms, about 34 ms to about 36 ms, about 36 ms to about 38 ms, or about 38 ms to about 40 ms.

With respect to the first and second voltages described herein, is understood that a first and a second voltage can be applied or occur in any order or configuration in a liquid crystal assembly described herein (e.g., whether the first or second is in open or closed state, has an applied voltage at 0V or +/− Voltage, as described herein). For example, the polarizers within the LC assembly similarly can be configured (e.g., oriented) to effect whether the first or second is in open or closed state, has an applied voltage at 0V or +/−a Voltage, as described herein. For example, when the polarizers are cross polarized, a voltage of 0 V may be applied to assume the open state (e.g., the LC cell rotates the polarization of the light transmitted from the first polarizer such that the light passes through the second polarizer). When the polarizers are cross polarized, a +/− Voltage may be applied to assume the closed state (e.g., the LC cell does not rotate the polarization of light transmitted from the first polarizer such that the light is blocked or attenuated by the second polarizer). For example, when the polarizers have the same polarization (e.g., same orientation), a +/− Voltage may be applied to assume the open state (e.g., the LC cell does not rotate the polarization of the light transmitted from the first polarizer and the light passes through the second polarizer). When the polarizers have the same polarization (e.g., same orientation), a voltage of 0 V may be applied to assume the closed state (e.g., the LC cell rotates the polarization of light transmitted from the first polarizer and the light is blocked or attenuated by the second polarizer).

According to one aspect of the LC shutter assembly 100, a dyestuff polarizer 104 is paired with a Twisted Nematic (TN) liquid crystal cell 106 that may be tuned to a desired separation between the visible and NIR spectral bands. When used with a TN cell as the LC cell 106, the dyestuff polarizer 104 enables high attenuation in the visible band with only approximately 15% attenuation in the ICG emission band when used in ICG fluorescence imaging. As such, the engineered dyestuff polarizer provides approximately 85% transmission of the ICG emission band regardless of whether the LC shutter 100 is in either the closed or opened state. This increased transmission of emission light is particularly useful when producing composite image(s) by superimposing visible light over the available NIR fluorescence.

### Tuning to Fluorescent Dyes

In numerous aspects, the cut off wavelength for the polarizer may be tuned or engineered to a specific wavelength that is optimized for the specific fluorescent molecule that is being subject to detection by the system utilizing the liquid crystal shutter assembly of the present invention. In such embodiments, the polarizer can be configured to have a light cut-off in a range adjusted to the emission range of the applicable fluorescent moiety. In some examples, the light cut-off range of the polarizer may comprise the longest wavelength of the visible spectral range. In some examples, the light cut-off range of the polarizer may be within the visible spectral range. The polarizer may be ineffective and transparent above the longest wavelength of the visible spectral range. In another example, the polarizer may be ineffective and transparent below the shortest wavelength of the visible spectral range. For example, the systems and methods of the present disclosure can be configured or optimized for fluorophores in the NIR range and/or for fluorophores outside the NIR range. For example, with an ICG dye, the polarizer can be configured to have a cut-off in a long wavelength spectral range applicable to the excitation light, for example between about 600 nm and about 850 nm, or between about 700 nm to about 800 nm, about 800 nm to about 950 nm, about 800 nm to about 880 nm, about 775 nm to about 795 nm, or about 785 nm. Also, with an ICG dye, the polarizer can be configured to have a wavelength cut-off in a range that can be configured to have a wavelength cut-off by 800 nm, or alternatively have a wavelength cut-off by 600 nm, 625 nm, 650 nm, 675 nm, 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm. The polarizer with an effective cutoff minimum enables emission band (e.g., from an excited fluorophore) to pass through unpolarized wavelengths to be collected, for example in the range of 800 nm to 880 nm, in instances where a polarizer cutoff occurs at 800 nm. In other embodiments, fluorophores and/or fluorochromes that emit light at wavelengths at or above the infrared range or, alternatively, emit light at wavelengths within the visible light range, or emit light at wavelengths below the visible light range may be used with engineered polarizers and LC shutters driven by voltages suitable to capture light at video switching rates based upon the corresponding emission spectra.

For example, the fluorescent molecule can comprise an ultraviolet (UV) dye, a blue dye, or both. Exemplary UV and blue dyes for fluorophores include: ALEXA FLUOR 350 and AMCA dyes (e.g., AMCA-X Dyes), derivatives of 7-aminocoumarin dyes, dialkylaminocoumarin reactive versions of ALEXA FLUOR 350 dyes, ALEXA FLUOR 430 (and reactive UV dyes that absorb between 400 nm and 450 nm have appreciable fluorescence beyond 500 nm in aqueous solution), Marina Blue and Pacific Blue dyes (based on the 6,8-difluoro-7-hydroxycoumarin fluorophore), exhibit bright blue fluorescence emission near 460 nm, hydroxycoumarin and alkoxycoumarin derivatives, Zenon ALEXA FLUOR 350, Zenon ALEXA FLUOR 430 and Zenon Pacific Blue, succinimidyl ester of the Pacific Orange dye, Cascade Blue acetyl azide and other pyrene derivatives, ALEXA FLUOR 405 and its derivatives, pyrene succinimidyl esters, Cascade Yellow dye, PyMPO and pyridyloxazole derivatives, aminonaphthalene-based dyes and dansyl chlorides, dapoxyl dyes (e.g., Dapoxyl sulfonyl chloride, amine-reactive Dapoxyl succinimidyl ester, carboxylic acid-reactive Dapoxyl(2-aminoethyl) sulfonamide), bimane dyes (e.g., bimane mercaptoacetic acid) and its derivatives, NBD dyes and its derivatives, QsY 35 dyes and its derivatives, fluorescein and its derivatives. The fluorescent molecule can comprise an infrared dye, near infrared dye or both. Exemplary infrared and near infrared dyes for fluorophores include: DyLight-680, DyLight-750, VivoTag-750, DyLight-800, IRDye-800, VivoTag-680, Cy5.5, or an indocyanine green (ICG) and any derivative of the foregoing, cyanine dyes, acradine orange or yellow, ALEXA FLUORs and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl) anthracene, 5,12-bis(phenylethynyl) naththacene, bisbenzimide, brainbow, calcein, carbodyfluorescein and any derivative thereof, 1-chloro-9,10-bis(phenylethynyl) anthracene and any derivative thereof, DAPI, DiOC6, DyLight Fluors and any derivative thereof, epicocconone, ethidium bromide, FLASH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′, 7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are known and described in international patent application no. PCT/US2014/056177, which is incorporated by reference herein in its entirety.

The fluorescent molecules used for detection of a sample by the systems and methods herein can comprise one or more dyes, two or more, three, four, five, and up to ten or more such dyes in a given sample using any class of dye (e.g., ultraviolet (UV) dye, a blue dye, an infrared dye, or near infrared dye) in any combination. In addition to fluorescence, the systems and methods disclosed are effective in photoluminescence applications. Bandgaps associated with Aluminum Nitride have similar emissions to visible spectrum fluorophores while bandgaps associated with Aluminum Gallium Arsenide have emissions similar to NIR fluorophores. Photoluminescence imaging has applications in, but not limited to, opto-electronics semiconductor processing as well as defect detection in organic and inorganic LED displays. Using the systems and methods herein, autofluorescence in an organ, organ substructure, tissue, target, cell, or sample can be detected.

Using Controlled Excitation Energy with A Liquid Crystal Shutter

According to various aspects, integrating an LC shutter, such as but not limited to the LC shutter 100, in conjunction with proper tuning of spectral filters, such as but not limited to the polarizer 104, into a wide variety of combined visible light and NIR light systems, enables NIR images to be captured without the overwhelming contributions of visible light. These improvements can be further enhanced when paired with techniques to reduce the effects of light pollution within the emission band being collected.

Fluorescent and photoluminescent photo pumping relies on a strong source to excite the material in question. In particular, increasing the excitation power can yield increased signal. This type of signal enhancement is effective up to the point of saturation of the fluorophore or non-radiative recombination losses of a photoluminescent junction.

Typically, increasing the excitation energy has the traditionally undesirable effect of adding heat to the sample being measured. The undesirable effects may be mitigated by reducing the duration of the excitation such that the average wattage incident on the measured sample is maintained below a pre-determined level or maintained at a safe level required for the application. For example, in in vivo applications where the systems and methods disclosed herein are used to image an organ, organ substructure, tissue, target, cell or samples, excitation energy imparted to the tissue must fall below known safety standards and thresholds to minimize heating and/or damage. In various aspects, controlling the peak intensity and duration of the excitation energy provides a controlled excitation energy. The controlled excitation energy may be further adjusted such that the signal to noise (SNR) of the NIR image can be enhanced while preserving boundaries due to damage thresholds or safety concerns.

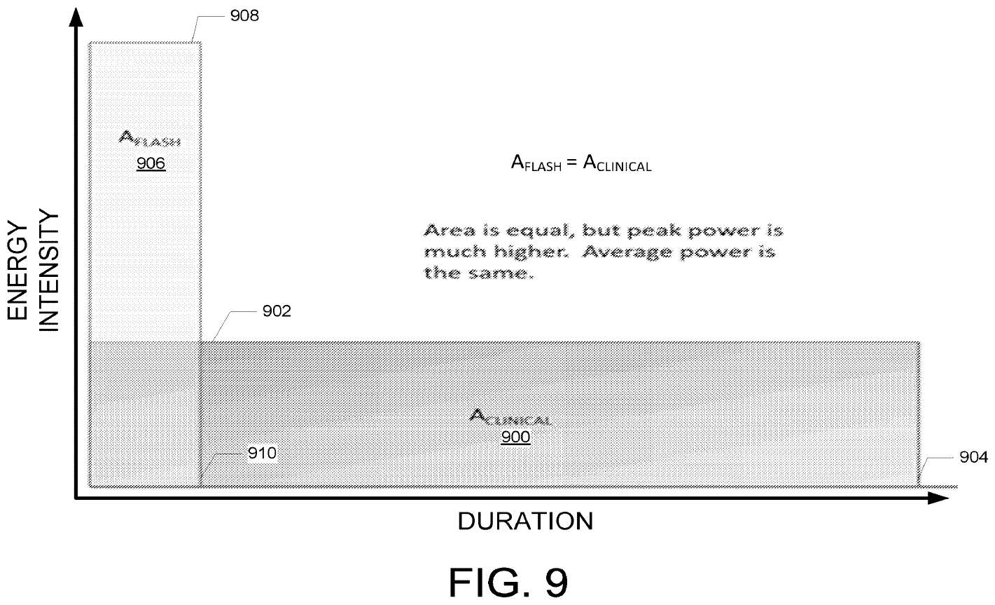

Referring to FIG. 9, patient safety standards may be quantified as the area under a curve plotting the energy imparted on tissue over time or duration. In physiologic applications, there is a safe level of energy that can enter the tissue under which tissue is not damaged. As shown, the area depicted under A_{CLINCAL} 900 illustrates an example of a “safe” exposure to energy (e.g., to living tissue) as indicated by the area under the curve formed by a threshold level or intensity of energy depicted by 902 which may be imparted for a short or long duration (e.g., for an extended amount of time). The present system can use higher intensity energy in short duration to achieve a safe level of energy or less which has advantage to enhance the photons available for imaging while still meeting or exceeding patient safety in clinical applications under which tissue is not damaged. As illustrated in FIG. 9, a burst controlled intense excitation energy identified as A_{FLASH} 906 illustrates an intense excitation energy pulse that may be applied for a short duration using the systems and methods disclosed herein. As shown, the area under the curve (A_{FLASH} 906) defined by the controlled higher intensity excitation energy indicated by 908 for a flash duration indicated by 910. As shown, although the flash energy 908 intensity is much higher than that of the clinical energy intensity 902, because the flash duration 910 is much shorter than the clinical duration 904, the area under the curve (AFLASH 906) is equal to or less than the area under the curve A_{CLINCAL} 900, which meets or exceeds the safety standards under which tissue is not damaged. As shown in FIG. 9, the area under the A_{FLASH} 906 curve is approximately equal to or less than the area under the A_{CLINCAL} 900 curve, such that the total energy imparted on the tissue by the controlled excitation energy falls below applicable safety thresholds. In certain embodiments used herein, the systems and methods of the present invention are configured (for example, by adjusting the intensity of the energy in and the duration of the excitation energy pulse) so that the area under the curve (AUC) for A_{FLASH} 906 is equal to or less than the AUC for A_{CLINCAL} 900.

It is understood that the methods described herein using controlled excitation energy can be applied to methods and systems using the LC shutter assemblies of the present disclosure, and/or with Blaze Bioscience systems.

In various aspects of the aforementioned methods, the liquid crystal assembly includes an engineered polarizer disposed in a path of light from the sample where the polarizer is configured to have an effective spectral range such that the long wavelength cut-off range is between 600 nm and 900 nm or about 800 to 880 nm. The method may further include directing the emission light and the reflected visible light through the liquid crystal assembly wherein the reflected visible light, having a wavelength within the effective spectral range of the engineered polarizers may be blocked or allowed to pass through the liquid crystal assembly depending on the state of the liquid crystal. The emission light incident on the liquid crystal, having a wavelength outside the effective spectral range of the engineered polarizer will pass mostly unattenuated, independent of the liquid crystal state.

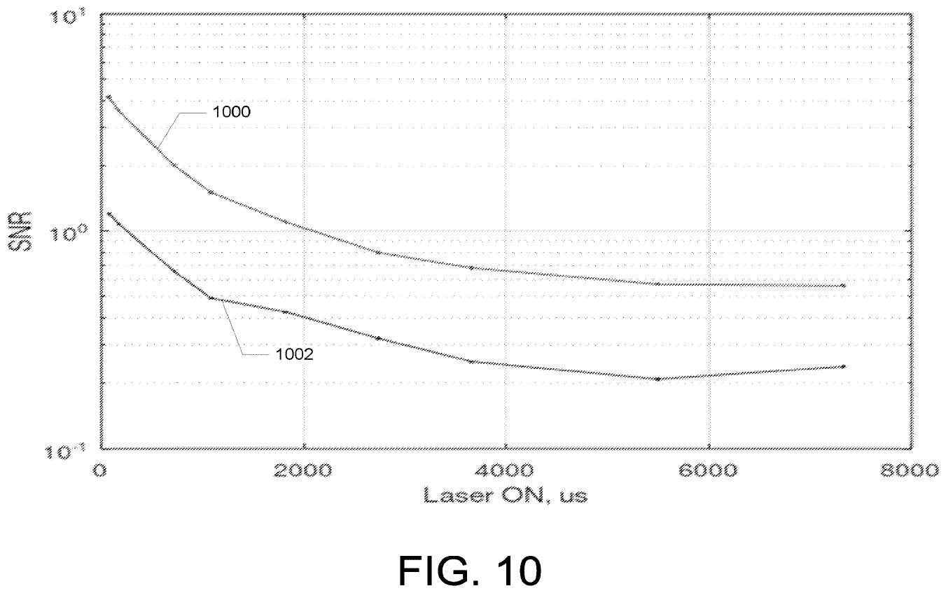

Furthermore, in specific applications of video rate imaging, the inherent sequencing of individual frames which are time multiplexed with a LC shutter is well paired with controlled and/or pulsed energy excitation. In some examples, the controlled and/or pulsed energy excitation may be white light, NIR light, IR light, or any other type of excitation light. Controlled excitation energy has the added advantage that in addition to creating a more intense excitation relative to ambient light pollution in the emission spectral band, controlled excitation energy increases the ratio of the signal to background or ambient light, allowing a shorter exposure time (i.e., video rate sampling duration). According to various aspects, the shorter exposure time permitted by use of the controlled excitation energy reduces the amount of thermal noise integrated into the image from the sensor and thus has the secondary benefit of improving the image SNR. In particular, the SNR increases significantly with a decreased laser pulse width and corresponding decrease in integration time. Referring to FIG. 10, the SNR for the pulsed or controlled excitation energy 1000 is greater than the SNR for a lower intensity energy pulse 1002. It is further shown, that as the laser time duration is shortened, the signal to noise continues to rise.

A type of SNR for image analysis is contrast-to-noise ratio (CNR). The CNR of an image may be improved when the object intended to be detected is bright and the surrounding areas are dark. When the object intended to be detected is fluorescence, the amount of light available is much lower than ambient light. Moreover, the visible illumination added to aid in detail work, such as surgical procedures, is often much brighter than typical ambient light. Therefore, the fluorescent light to be detected may be orders of magnitude below that of the ambient background light and the CNR of the fluorescent image is below a useable range. To improve CNR, fluorescent light may be increased. The two known ways to increase fluorescent light are increasing the amount of fluorescent material or increasing the excitation energy. However, increasing the amount of fluorescent material may not be viable in biologic material due to physiological barriers. Furthermore, increasing the amount of excitation energy may not be viable due to the potential of thermal damage to the biologic material.

Traditionally, CNR has been improved by reducing the background light (e.g., visible light) such that the fluorescent light is more apparent. Ideally, the source of the background light is removed or turned off intermittently. When the source of the background light cannot be removed or turned off, the excitation source is modulated, and dark frames are collected for subtraction of the background light from the signal. Using this method results in the detection of fluorescent light at the cost of the bulk of the camera dynamic range being filled with the bright background light which must be removed through subtraction. The images have a high level of noise and require long sequence times, resulting in reduced video frame rates.

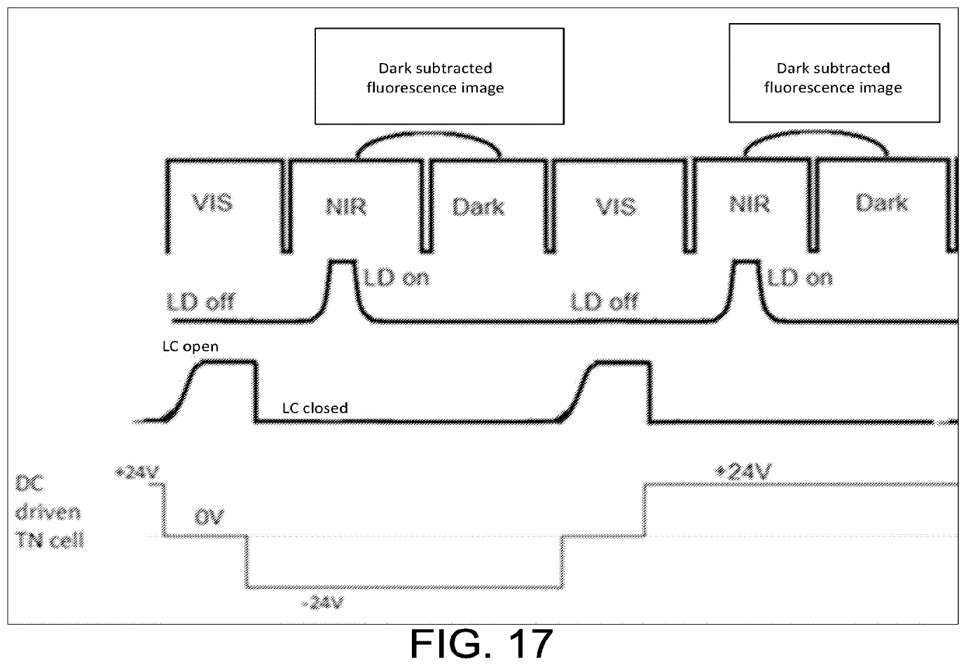

Although the frame subtraction system is an improvement on prior systems, reducing the sequence times (e.g., the necessary video rate sampling duration) through use of the LC shutter 100 may significantly improve video frame rate, block the interference light (e.g., background light), improve and increase CNR, and enhance SNR in bright ambient light settings. The enhanced CNR and/or SNR may enable a fast or increased frame rate. An LC shutter 100 may be used to block the background light intermittently, as shown in FIG. 17. When the LC shutter is in the open state (e.g., a first voltage of 0 V is applied), the bright background light (e.g., reflected visible light from a sample) is allowed to pass through the LC shutter for imaging of the visible light (e.g., first video rate sampling duration). Imaging the visible light may be conducted during a first video rate sampling duration (e.g., when the LC shutter is in the open state). In some examples, the first video rate sampling duration (i.e., first exposure time) may be about 1 ms to about 2 ms, about 2 ms to about 3 ms, about 3 ms to about 4 ms, about 4 ms to about 5 ms, about 5 ms to about 6 ms, about 6 ms to about 7 ms, about 7 ms to about 8 ms, about 8 ms to about 9 ms, or about 9 ms to about 10 ms. In one example, the first video rate sampling duration may be about 2 ms to about 6 ms.

When the LC shutter is in the closed state (e.g., a second voltage is applied), the bright background light (e.g., visible light) may be blocked from the camera, allowing only NIR to pass and allowing imaging of the fluorescent light from the sample for a second video rate sampling duration (e.g., second exposure time). A laser diode may be turned on providing excitation energy to the fluorophore to allow imaging of the fluorescent light (i.e., emission light from the fluorophore) while the LC shutter 100 is in a closed state. In some examples, the second video rate sampling duration (i.e., second exposure time) may be about 5 ms to about 6 ms, about 6 ms to about 7 ms, about 7 ms to about 8 ms, about 8 ms to about 9 ms, about 9 ms to about 10 ms, about 10 ms to about 11 ms, about 11 ms to about 12 ms, about 12 ms to about 13 ms, about 13 ms to about 14 ms, about 14 ms to about 15 ms, about 15 ms to about 16 ms, or about 16 ms to about 17 ms. In some examples, the second video rate sampling duration may be about 12 ms to about 16 ms or about 13 ms to about 17 ms.

After the imaging of fluorescent light, the laser diode may be turned off for a short period of time (e.g., third video rate sampling duration) while the LC shutter remains in a closed state to allow imaging of a dark background image. When the laser diode is turned off, the laser diode stops providing excitation energy. In some examples, the third video rate sampling duration (i.e., third exposure time) may be about 5 ms to about 6 ms, about 6 ms to about 7 ms, about 7 ms to about 8 ms, about 8 ms to about 9 ms, about 9 ms to about 10 ms, about 10 ms to about 11 ms, about 11 ms to about 12 ms, about 12 ms to about 13 ms, about 13 ms to about 14 ms, about 14 ms to about 15 ms, about 15 ms to about 16 ms, or about 16 ms to about 17 ms. In some examples, the third video rate sampling duration may be about 12 ms to about 16 ms or about 13 ms to about 17 ms.

In an aspect, the dark background image may be subtracted from the fluorescent image providing a clear fluorescent image. The LC shutter 100 in a closed state blocks any light outside of the emission band and spectrally blocks interfering light (bright background light) from being captured. Removing the bright background light (e.g., visible light) allows for higher analog gains to be used during image capture to fill the camera dynamic range. Increasing the camera gain allows for much shorter integration times. In this embodiment, the LC shutter 100 acts as a momentary spectral filter allowing the viewing of fluorescence when in the closed state and viewing of visible light when in the open state. The reduced integration times needed for a minimum CNR allow for real time video of fluorescence images even in the presence of bright background light.

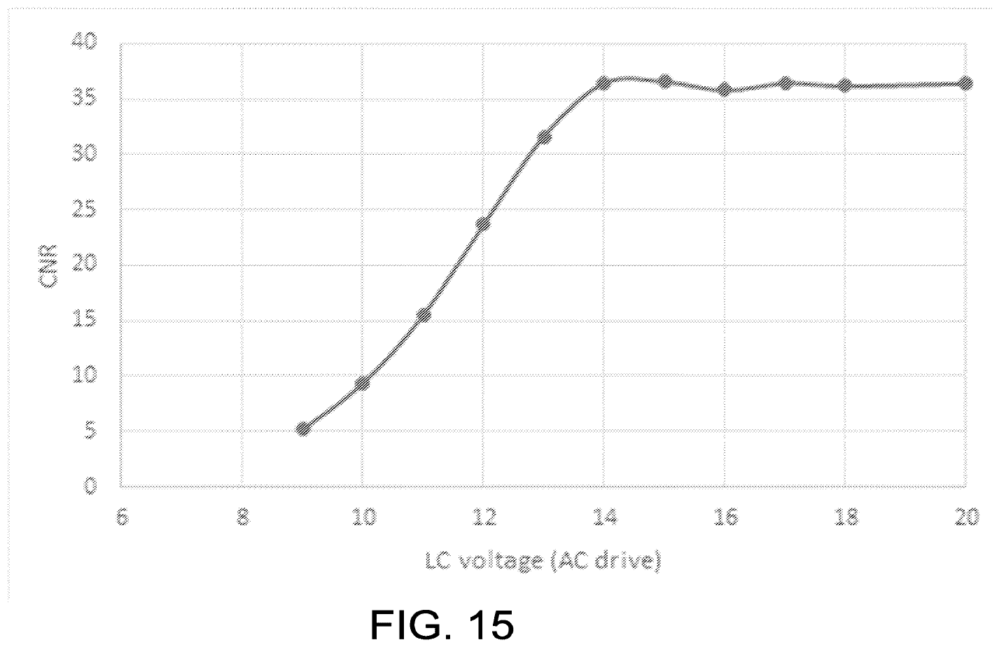

As shown in FIG. 8, the blocking of visible light is increased with increasing drive voltage to the LC media 106. As the visible light is blocked, the fluorescent image buried under the visible image is revealed. The CNR values of the fluorescent image increase with increasing drive voltage to the LC media allowing better imaging of the fluorescent image, as shown in FIG. 15.

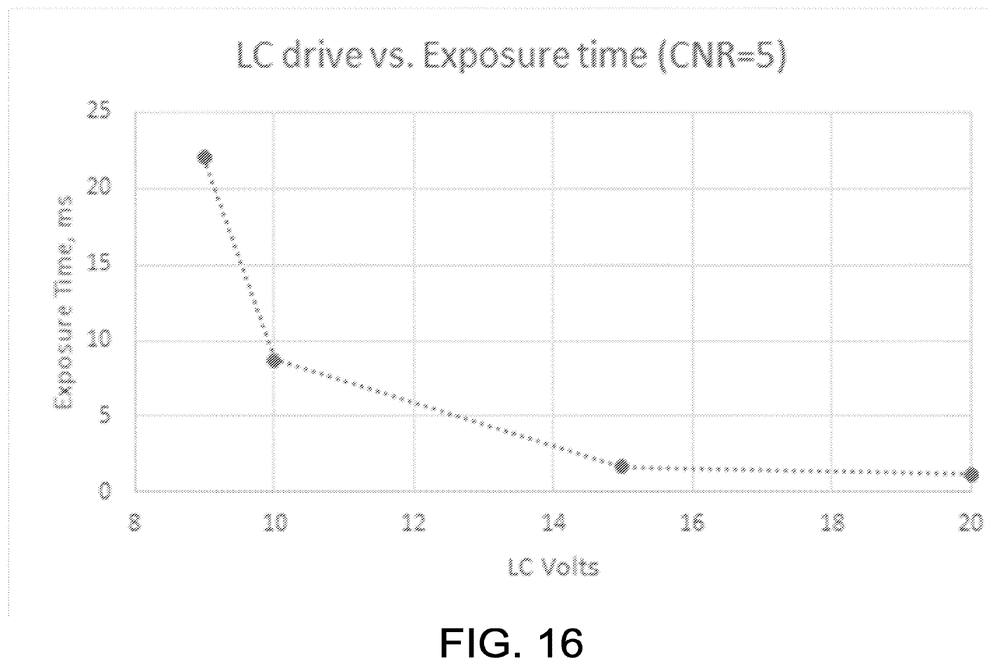

The increased CNR achieved by using the LC shutter 100 allows for post processing of the fluorescent image by digital gain, as opposed to requiring longer exposure times. The post processing via digital gain and shorter exposure times results in increased frame rates of the fluorescent image. As shown in FIG. 16, the exposure time necessary to maintain a CNR of 5 decreases as voltage increases. Exposure times of less than 5 milliseconds can be achieved when supplying a voltage of about 13 V or higher to the LC shutter 100.

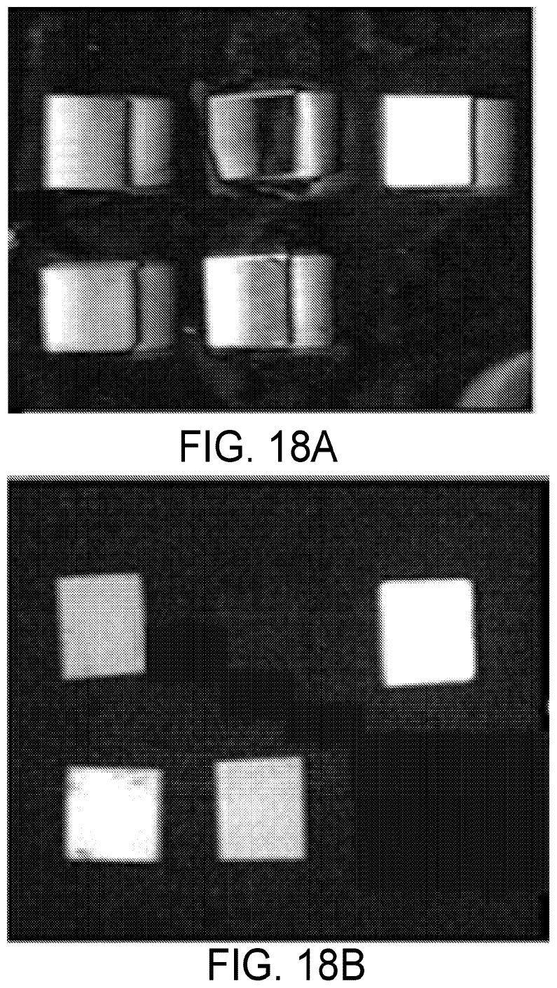

As shown in FIG. 18A-B, the reduced brightness of the dark image with the LC shutter assembly in the closed state reduces artifacts of the subtracted image as described herein. Additionally, the reduced exposure time (i.e., video rate sampling duration) of the dark frame (laser off) minimize motion blur in the images. Furthermore, the use of the LC shutter assembly reduces the intensity of the dark frame and the ratio of the dark frame to the NIR frame due to the reduced brightness of the dark image. As shown in FIG. 18A, fluorescent images taken on a linear stage (e.g., being rotated or set in motion) using bright background subtraction resulted in images having unclear boundaries due to the subtracted image showing errors where the dark image was translated relative to the NIR image. Additionally, in FIG. 18A, the longer dark frame exposure time results in an image that appears to capture the motion and is not an accurate depiction of the locations of the fluorescence. As shown in FIG. 18B, fluorescent images taken on a linear stage (e.g., being rotated or set in motion) using the LC shutter 100 to block the visible light resulted in images having clear boundaries. By utilizing the LC shutter 100 to block visual light and shorten exposure times, clear fluorescent images may be produced providing exact locations of fluorescence. Clear fluorescent images are vitally important in surgical procedures where surgeons need exact locations of various components of the body to conduct safe, efficient, and effective surgeries.

### Using a Liquid Crystal Shutter to Reduce Ghosting or Undesired Light

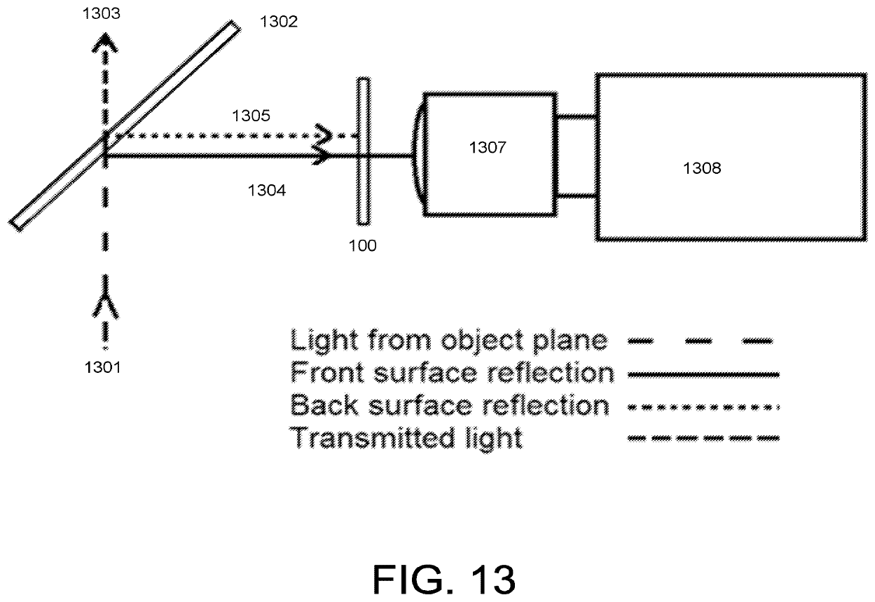

As shown in FIG. 13, imaging systems may include a beam splitter 1302. The function of the beam splitter is to split the incoming light into a reflected and a transmitted beam. One undesirable feature of certain types of beam splitters is the creation of a secondary reflection. The secondary reflection may create a “ghost” image. As shown in FIG. 13, light from the object plane 1301 is partially reflected off the front surface of the beam splitter 1302 to make a front surface reflection 1304. The back surface of the beam splitter will make a back surface reflection 1305 (e.g., “ghost” image). The rest of the light is transmitted light 1303 without a “ghost” image. Both the front surface reflection 1304 and the back surface reflection 1305 of the beam splitter 1302 have S dominant polarizations relative to the orientation of the beam splitter surfaces. Conventionally, a back surface coating will attempt to minimize the back surface reflection 1305 with an anti-reflection (AR) coating while a front surface coating will create the front surface reflection 1304 (i.e., primary reflection) of the beam splitter 1302. Conventionally, the front surface reflection 1304 must have a reflectivity that is at least 10 times that of the back surface reflection 1305 to render the back surface reflection 1305 un-noticeable or “invisible” relative to the front surface reflection 1304. In other words, having a 10-fold higher front reflectivity (e.g., reflectivity of the front surface reflection 1304) than the back reflectivity (e.g., reflectivity of the back surface reflection 1305), or a 10:1 ratio (of front reflection/back reflection), is considered a desirable outcome to reduce ghosting in a system containing a beam splitter 1302. It is technically difficult to suppress the S polarized reflections and achieve a back surface reflectivity (e.g., reflectivity of the back surface reflection 1305) below 1% across the entire visible spectrum, thus intentionally increasing the reflectivity of the front surface reflection 1304 is the only mechanism to achieve a 10:1 reflectivity ratio. This limits the minimum total reflectivity (by adding both the front and back percentage reflection) of the beam splitter to 11%. In systems which are attempting to keep very low total reflectivity, this 11% lower bound is undesirable.

In contrast to S polarizations, it is much easier to lower and control the P polarization of either the front surface reflection 1304 or the back surface reflection 1305 to near zero values. The LC shutter disclosed offers an advantage in that only linearly polarized light is passed through the shutter in the effective spectral range of the polarizers. Thus, the LC shutter can be oriented such that it will block the S polarized light reflected off the beam splitter. By orienting the polarizers of the LC shutter to block the S polarized light, only the P polarized light is imaged. The front surface and back surface coatings of the beam splitter may be configured to minimize the S polarized reflections on both the front and back surfaces of the beam splitter. By adding a small amount of P polarized reflection only on the front surface coating, a 10:1 or greater reflectivity ratio can easily be achieved using the described LC shutter while imaging only the P polarized light. The S polarized light is then simply engineered to the lowest reasonable levels. By properly designing the coatings, the front surface reflection 1304 to back surface reflection 1305 ratio in P polarization can exceed 10:1 while keeping the total reflectivity of the beam splitter 1302 below the 11% minimum. The front to back ratio in S polarization will not achieve a 10:1 ratio, but this light is subsequently filtered out with the properly oriented polarizing filters of the LC shutter in the present disclosure. The first polarizing film 1101(a) of the LC shutter 100 may be oriented to block the S polarized light from both the back surface reflection 1305 and front surface reflection 1304 and pass the P polarized light only. The intensity ratio of the front surface reflection 1304 and the back surface reflection 1305 can be increased with the P polarized light being passed to the imaging lens 1307 and then to the camera 1308. By removing the S polarized light, and imaging only in P polarized (relative to the angle of the beam splitter) light, a variety of front to back reflection ratios can be achieved in applications by engineering the reflections of P polarized light in instances when low reflectivity values are desired.

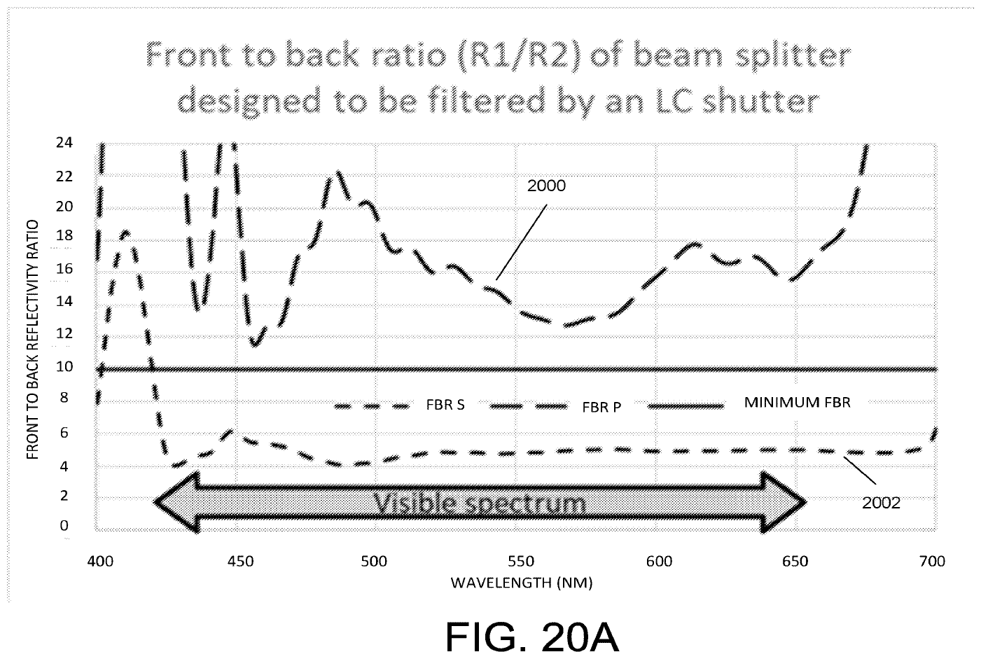

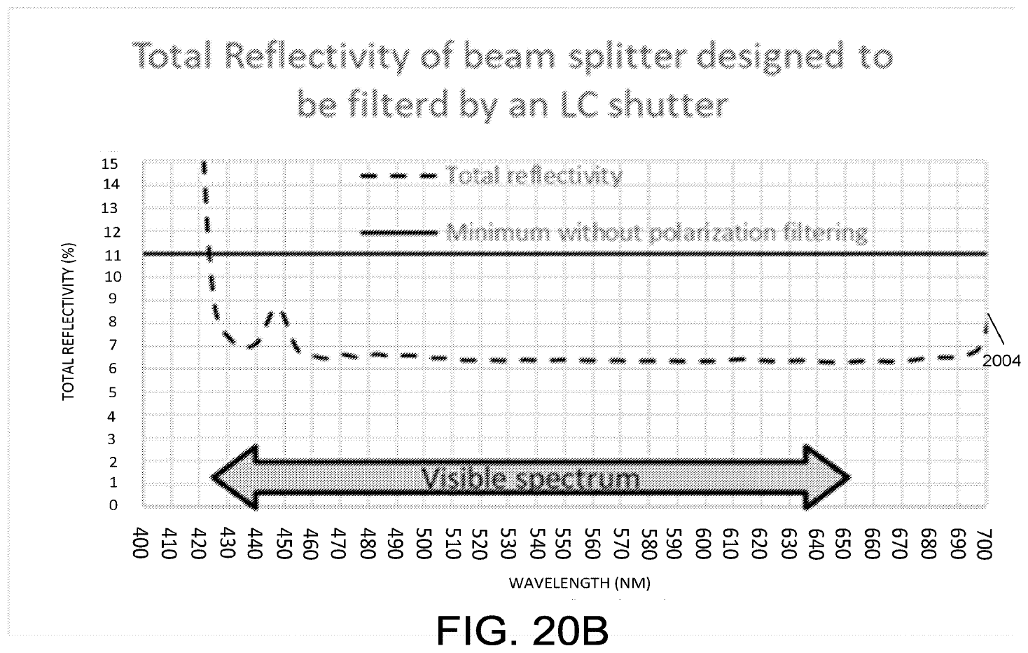

In some aspects, front to back reflection ratios of at least a 2:1, at least a 4:1, at least a 6:1, at least a 8:1, at least a 10:1, at least 11:1, at least a 12:1, at least 14:1, at least a 15:1, at least 16:1, at least a 17:1, or at least a 20:1 front reflection/back reflection ratio inclusive (i.e., for any ratio between 2:1 and 20:1) can be achieved accordingly, any of which are effective for reducing ghosting. This technique of designing beam splitter coatings to leverage off of the linear polarized light is an advantage of the LC shutter that can cut or reduce total reflectivity of the beam splitter 1302 well below the 11% nominal minimum achievable when no polarizer is used. The system can be configured to a total reflectivity of the beam splitter of 11% or lower, 10% or lower, 9% or lower, 8% or lower, 7% or lower, 6% or lower, 5% or lower, 4% or lower, or 3% or lower. FIG. 20A exemplifies an example of front to back reflectivity ratio (FBR) of approximately 10:1 over the visible spectrum. As shown in FIG. 20A, the P polarized light FBR 2000 can maintain a FBR of 10:1 or greater throughout the visible spectrum. As shown in FIG. 20A, the S polarized FBR 2002 is below the desired FBR of 10:1 throughout the visible spectrum, however, the S polarized light is attenuated or blocked by the first polarizing film 1101(a). As shown in FIG. 20B, using the LC shutter to filter out the S polarized light from the beam splitter can result in a total reflectivity of below 11% throughout the visible spectrum.

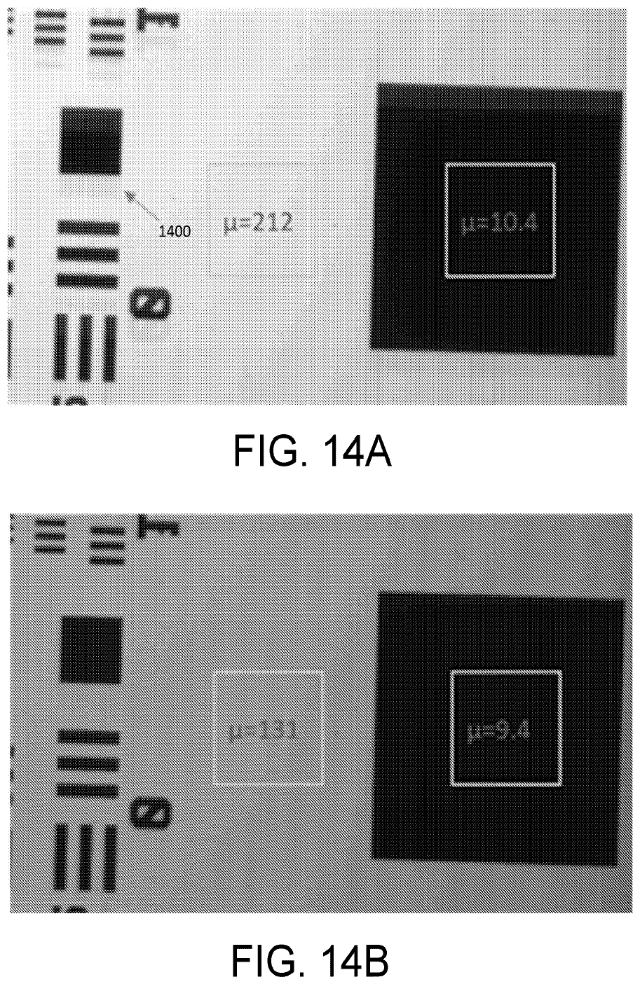

As shown in FIG. 14B, removing the S polarized light from the back surface reflection 1305 can remove the “ghost” image 1400. As shown in FIG. 14A, when the LC media 106 is oriented such that the first polarizer attenuates P polarized light, a ghost image 1400 may be present on the lower edges of the image. As shown in FIG. 14B, when the LC media 106 is oriented such that the first polarizer attenuates S polarized light, the ghost image is removed. In some examples, the first polarized film 1101(a) may be engineered such that the S polarized light is 100% attenuated or blocked. By blocking the S polarized light and engineering the beam splitter to have a 10:1 or greater reflectivity ratio (front surface reflection to back surface reflection) of P polarized light, no ghost image is present or is reduced significantly, resulting in a clearer image. In other examples, the first polarizing film 1101(a) can be oriented to block other types of polarized light, such that undesired polarized light can be filtered before being transmitted through the liquid crystal shutter assembly. As illustrated in FIGS. 14A-B, the removal of the ghost image results in less signal intensity (e.g., signal intensity drops from μ=212 to μ=131) due to the S reflections being attenuated. The resulting image may lose some intensity; however, the image also defines clear boundaries without a “ghost” image 1400 which is of vital importance when detailed imaging is required.

In some embodiments, the polarizers 104 of the LC shutter 100 may be configured to improve image quality in any situation where a desired light can be made to have a different polarization than undesirable light. In an example, a laser illuminator may be used in an imaging system. The laser illuminator may produce light that is heavily polarized. In some examples, it may be beneficial to block out laser light to allow viewing of other light, such as fluorescent light. Typically, the light from a laser illuminator is blocked using spectrally specific filters. Using polarization can help assist blocking laser light given that the polarizers have at least partial efficacy at the lasing wavelength. In some examples, the typical spectral filters may be configured to block polarized laser light and only allow fluorescent light to be viewed. The typical spectral filters may be configured to block out polarized laser light outside of the fluorescent range desired and allow fluorescent light to be preferably viewed at specific wavelengths tuned to a fluorescent molecule. For example, such configurations can be used to visualize a fluorescent molecule described herein, including a fluorescent molecule or moiety in the NIR spectrum from between 650 and 850 nm, or 600 nm and about 850 nm, or between about 700 nm to about 850 nm, about 800 nm to about 950 nm, about 800 nm to about 880 nm, about 775 nm to about 795 nm, or about 785 nm, 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, or 900 nm. For example, the first polarizing film 1101(a) may be configured to block polarized laser light having a wavelength of about 780 nm to about 790 nm, while allowing emission light from a fluorescent molecule to pass through with minimal attenuation. It is understood that a variety of laser illuminators may be used in the systems including gas lasers, solid-state lasers, semiconductor lasers, chemical lasers, dye lasers, metal-vapor lasers, or other types of lasers. It is understood that the first polarizing film 1101(a) may be oriented such that different undesirable polarized lights from various laser illuminators may be blocked. Further, any undesired polarized light may be blocked by adjusting the orientation of the first polarizing film 1101(a).

The first polarizing film 1101(a) and second polarizing film 1101(b) may be configured to block heavily polarized laser light while allowing fluorescent light to pass through the LC shutter 100, improving the signal-to-noise ratio of the resulting fluorescent image. In an aspect, the first polarizing film 1101(a) and the second polarizing film 1101(b) may be configured to be effective in the wavelength of the light from the laser illuminator. In an example, the first polarizing films 1101(a) may be configured to have an effective range from 420 to 790 nm and thus be effective in helping to block laser light of 780 to 790 nm.

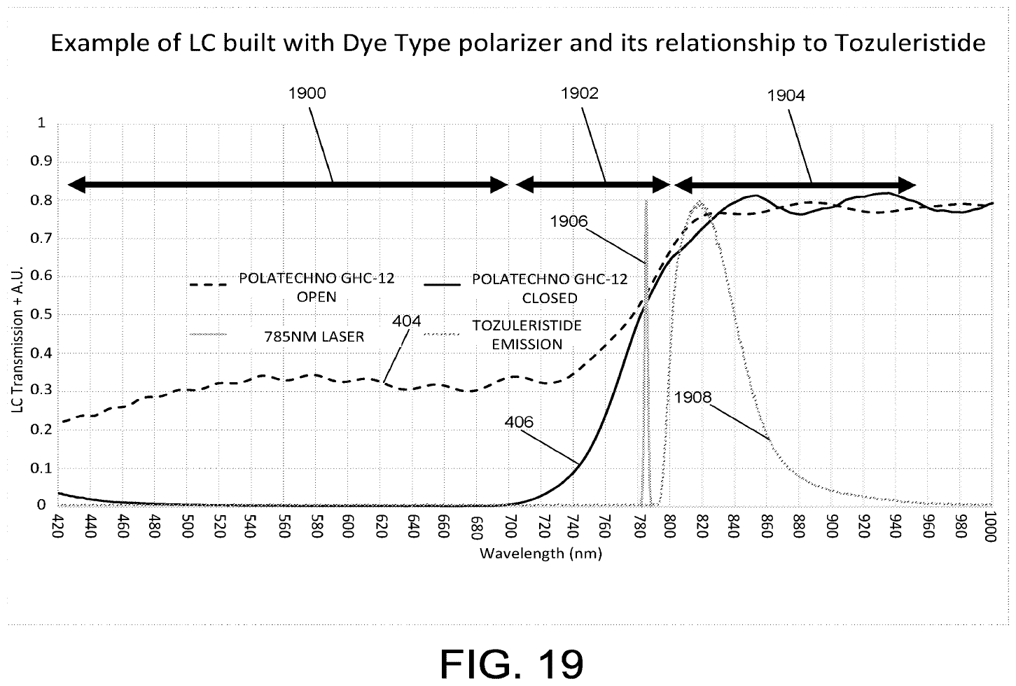

FIG. 19 shows using a liquid crystal shutter assembly described herein configured to visualize a specific peptide-fluorophore conjugate (tozuleristide). In this example, the liquid crystal shutter assembly is configured to block the heavily polarized light of the laser illuminator using the first polarizing film 1101(a). The first polarizing film 1101(a) may be effective in the visible light spectrum 1900 and a portion of the NIR spectrum 1902. To block the heavily polarized laser light 1906 light from the laser illuminator, the first polarizing film 1101(a) is oriented to attenuate the heavily polarized laser light 1906 from the laser illuminator. The heavily polarized laser light 1906 may have a wavelength of about 780 nm to about 790 nm. The first polarizing film 1101(a) may be configured to be effective up to about 800 nm and then lose effectiveness at wavelengths over 800 nm. By blocking the heavily polarized laser light 1906 visualization of the fluorescent molecule (e.g., peptide-fluorophore conjugate (tozuleristide)) at wavelengths of about 800 nm to 840 nm is optimized. Regardless of the state of the liquid crystal shutter assembly, fluorescence may be viewed since the fluorescent light 1908 is outside the light cut-off (e.g., effective range) of the polarizers.

As illustrated in FIG. 19, visible light (e.g., wavelengths from about 420 nm to 700 nm) can be fully attenuated (e.g., blocked) by the liquid crystal shutter assembly in the closed state 406. In the open state 404, the liquid crystal shutter assembly may be configured to allow visible light to pass through the liquid crystal shutter assembly at about 50% attenuation, allowing for imaging of visible light. In the visible light spectrum 1900, polarized undesired light (e.g., ghost images from S polarized light off a beam splitter, as described herein) may be attenuated or blocked by orienting the first polarizing film 1101(a) of the first polarizer to block the polarized undesired light. Blocking or attenuating undesired polarized light during visible light imaging can result in clearer visible images (e.g., reducing shadows or ghost images).

### Detection Methods

In another aspect, disclosed herein is a method for imaging a sample, comprising: emitting, by a light source, infrared or near infrared light to induce fluorescence from a sample; directing, by a plurality of optics, the infrared or near infrared light to the sample; receiving, by the plurality of optics, the fluorescence from the sample at a detector, wherein the infrared or near infrared light is directed to the sample substantially coaxially with fluorescence light received from the sample in order to decrease shadows; and forming a fluorescence image of the sample and a visible light image of the sample on the detector. In some embodiments, the method herein comprises using the imaging system disclosed herein. In some embodiments, the sample is an organ, organ substructure, tissue or cell. In some embodiments, the method of imaging an organ, organ substructure, tissue or cell, comprises imaging the organ, organ substructure, tissue or cell with an imaging system herein. In some embodiments, the method further comprises detecting a cancer or diseased region, tissue, structure or cell. In some embodiments, the method further comprises performing surgery on the subject. In some embodiments, the method further comprises treating the cancer. In some embodiments, the method further comprises removing the cancer or the diseased region, tissue, structure or cell of the subject. In some embodiments, the method further comprises imaging the cancer or diseased region, tissue, structure, or cell of the subject after surgical removal. In some embodiments, the detecting is performed using fluorescence imaging. In some embodiments, the fluorescence imaging detects a detectable agent, the detectable agent comprising a dye, a fluorophore, a fluorescent biotin compound, a luminescent compound, or a chemiluminescent compound. In some embodiments, the detectable agent comprises any one or more fluorophore described herein. In some embodiments, the detectable agent comprises an ICG or tozuleristide.

In another aspect, as disclosed herein is a method of treating or detecting in a subject in need thereof the method comprising administering a companion diagnostic, therapeutic agent, or imaging agent, wherein the companion diagnostic or imaging agent detected by the systems and methods described herein. In another embodiment, the method of administering a companion diagnostic comprises any one of the various methods of using the systems described herein. In another embodiment, the diagnostic or imaging agent comprises a chemical agent, a radiolabel agent, radiosensitizing agent, fluorophore, an imaging agent, a photosensitizing agent, sonosensitizing agent, a diagnostic agent, a protein, a peptide, a nanoparticle or a small molecule. In another embodiment, the system incorporates radiology or fluorescence, including the X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), surgical instrument, operating microscope, confocal microscope, fluorescence scope, exoscope, or a surgical robot. In another embodiment, the systems and methods are used to detect a therapeutic agent or to assess the agent's safety and physiologic effect. In yet another embodiment, the safety and physiologic effect detected by the systems and methods is the agent's bioavailability, uptake, concentration, presence, distribution and clearance, metabolism, pharmacokinetics, localization, blood concentration, tissue concentration, ratio, measurement of concentrations in blood and/or tissues, assessing therapeutic window, range and optimization.

In various aspects, the imaging system disclosed herein can be used in a method of imaging abnormal tissue, cancer, tumors, vasculature, or structure in a fluorophore from a subject. In an aspect, the method includes producing an image of the vasculature or structure by imaging fluorescence using the imaging system provided herein. In another aspect, the method includes producing an image of the abnormal tissue, cancer, tumor, vasculature, or structure by imaging fluorescence using the imaging system comprising the liquid crystal assembly systems and methods disclosed herein. In some examples, the fluorescence imaged may be autofluorescence, a contrast or imaging agent, chemical agent, a radiolabel agent, radiosensitizing agent, photosensitizing agent, fluorophore, therapeutic agent, an imaging agent, a diagnostic agent, a protein, a peptide, a nanoparticle, or a small molecule, or any combination thereof or any combination thereof. In some examples, the method can further include administering the contrast or imaging agent to the subject. In some examples, the contrast or imaging agent may be a dye, a fluorophore, a fluorescent biotin compound, a luminescent compound, a chemiluminescent compound, or any combination thereof.

In various aspects, the contrast or imaging agent further includes a protein, peptide, amino acid, nucleotide, polynucleotide, or any combination thereof. In some examples, the contrast or imaging agent may be tozuleristide. In some examples, the contrast or imaging agent can absorb a wavelength between about 200 nm to about 900 nm. In some examples, the contrast or imaging agent may include DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, rythrosine, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin, coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl(2-aminoethyl) sulfonamide), a bimane dye, bimane mercaptoacetic acid, an NBD dye, a QsY 35, or any combination thereof.

In various aspects, administering the contrast or imaging agent to a subject may include intravenous administration, intramuscular administration, subcutaneous administration, intraocular administration, intra-arterial administration, peritoneal administration, intratumoral administration, intradermal administration, or any combination thereof.

In another aspect, the imaging can include tissue imaging, ex vivo imaging, intraoperative imaging, or any combination thereof. In some examples, the sample may be an in vivo sample, an in situ sample, an ex vivo sample, or an intraoperative sample. In another example, the sample may be an organ, an organ substructure, a tissue, or a cell. In some examples, the sample autofluoresces or exhibits autofluorescence. In an example, the autofluorescence may include an ocular fluorophore, tryptophan, or protein present in a tumor or malignancy. In some examples, the method may be used to visualize vessel flow or vessel patency.

In further aspects, the abnormal tissue, cancer, tumor, vasculature, or structure may include a blood vessel, lymph, vasculature, neuronal vasculature, or CNS structure. In some examples, the imaging may be an angiography, arteriography, lymphography, or cholangiography. In an example, the imaging may include detecting a vascular abnormality, vascular malformation, vascular lesion, organ or organ substructure, cancer or diseased region, tissue, structure, or cell. In further examples, the vascular abnormality vascular malformation, or vascular lesion may be an aneurysm, an arteriovenous malformation, a venous malformation, a lymphatic malformation, a capillary malformation, a mixed vascular malformation, a spinal dural arteriovenous fistula, or a combination thereof.

In various aspects, the organ or organ substructure may be a brain, heart, lung, kidney, liver, breast, skin, or pancreas. In some aspects, the method may include performing surgery on the subject. In an example, the surgery may be the surgery comprises angioplasty, cardiovascular surgery, aneurysm repair, valve replacement, aneurysm surgery, arteriovenous malformation or cavernous malformation surgery, venous malformation surgery, lymphatic malformation surgery, capillary telangiectasia surgery, mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or diseased region, tissue, structure or cell of the subject, or any combination thereof. In some examples, the imaging may include imaging a vascular abnormality, cancer or diseased region, tissue, structure, or cell of the subject after surgery. In some aspects, the method may include treating a cancer in the subject.

In various aspects, the method may include repair of an intracranial CNS vascular defect, a spinal CNS vascular defect; peripheral vascular defects; removal of abnormally vascularized tissue; ocular imaging and repair; anastomosis; reconstructive or plastic surgery; plaque ablation or treatment or restenosis in atherosclerosis; repair or resection (including selective resection), preservation (including selective preservation), of vital organs or structures such as nerves, kidney, thyroid, parathyroid, liver segments, or ureters; identification and management (sometimes preservation, sometimes selective resection) during surgery; diagnosis and treatment of ischemia in extremities; or treatment of chronic wounds.

### Adaptation of the LC Assembly to Microscopes

In some embodiments, the imaging system herein is stereoscopic. In some embodiments, the imaging system herein is not stereoscopic. In some embodiments, the imaging system herein is a surgical microscope, confocal microscope, fluorescence scope, exoscope, endoscope, or a surgical robot or an add-on (such as an imaging head) to any of the foregoing.

In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G); PROVido system, ARvido system, GLOW 800 system, Leica ARveo, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D October 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., RetinaVue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPAL1 NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

Combining or integrating a system herein into an existing surgical microscope, confocal microscope, fluorescence scope, exoscope, endoscope, or a surgical robot can be accomplished by: adapting an add on containing the imaging system, co-housing (in whole or in part), combining one or more aspect or component of the disclosed systems into the existing system, or integrating one or more aspect or component of the disclosed systems into the existing system. Such a combination can enhance image clarity and clarify image boundaries; enhance video rate and real-time imaging; reduce shadowing, motion effects, and/or ghosting; utilize improvements, enhance imaging, increase image clarity, optimize imaging, and improve surgical workflow, amongst other features of the systems and methods disclosed herein. Further such a combination or integration can utilize the LC shutter assembly, LC cell, polarizers, the first voltage and second voltage toggle for the liquid crystal between an open state and a closed state, light cut-off criteria, or any other feature of the systems disclosed herein, or any combination thereof.

The systems herein have been successfully adapted to one or more surgical microscope(s), confocal microscope, fluorescence scope, exoscope, endoscope, or surgical robot through use of an optical gasket used to create a light-tight interface between two mating optical mounts, mounting hardware, and fixtures to enable light passing from the applicable existing device to the systems described herein. For example, the systems herein have been successfully adapted to at least one KINEVO system (e.g., KINEVO 900), OMPI PENTERO system (e.g., PENTERO 900, PENTERO 800), using such interfaces, and could potentially successfully adapt to other Zeiss systems or Leica FL800, amongst others, using such interfaces.

Although certain embodiments, aspects, and examples are provided in the foregoing description, the inventive subject matter extends beyond the specifically disclosed embodiments or aspects to other alternative embodiments, aspects, and/or uses, and to modifications and equivalents thereof. Thus, the scope of the claims appended hereto is not limited by any of the particular embodiments or aspects described below. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures, systems, and/or devices described herein can be embodied as integrated components or as separate components.

What is claimed:

1. A liquid crystal assembly for use with an imaging system having a light source, the assembly comprising:
a polarizer disposed in a path of light from the light source; the polarizer configured to have a light cut-off in a range outside of its range of polarization;
a liquid crystal disposed in a path of light transmitted through the polarizer; and
a drive voltage source configured to apply a first voltage and a second voltage to the liquid crystal.

2. The liquid crystal assembly of claim 1, wherein the first voltage and second voltage achieve high and low electric fields across the liquid crystal.

3. The liquid crystal assembly of claim 1 or 2, wherein the first voltage and the second voltage toggle the liquid crystal between an open state and a closed state.

4. The liquid crystal assembly of any one of claims 1-3, wherein applying one of the first voltage or second voltage toggles the liquid crystal to the open state.

5. The liquid crystal assembly of any one of claims 1-4, wherein applying one of the first voltage or the second voltage toggles the liquid crystal to the closed state.

6. The liquid crystal assembly of any one of claims 1-5, wherein the polarizer is configured to be transparent above the light cut-off.

7. The liquid crystal assembly of any one of claims 1-6, wherein the light cut-off comprises a longest wavelength of a spectral range and the polarizer is configured to be ineffective and transparent above the longest wavelength.

8. The liquid crystal assembly of any one of claims 1-7, wherein the liquid crystal comprises a Twisted Nematic (TN) cell, Pi cell, thermotropic liquid crystal, and lyotropic liquid crystal, liquid crystal display (LCD), liquid crystal with photoconductor properties, or liquid crystal receptive to development and tuning under an electric field.

9. The liquid crystal assembly of any one of claims 1-8, wherein the second voltage induces a backflow in the liquid crystal.

10. The liquid crystal assembly of any one of claims 1-9, wherein one of the first voltage or second voltage is 0 V.

11. The liquid crystal assembly of any one of claims 1-10, wherein the second voltage is a plurality of voltages comprising a bi-polar square wave with an average value of 0 V.

12. The liquid crystal assembly of any one of claims 1-11, wherein the first voltage, or the second voltage, or both the first voltage and the second voltage, comprises a plurality of voltages creating a bi-polar square wave with an average value of 0V.

13. The liquid crystal assembly of any one of claims 11-12, wherein the bi-polar square wave comprises an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

14. The liquid crystal assembly of any one of claims 1-13, wherein the second voltage is a bi-polar square wave having an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

15. The liquid crystal assembly of any one of claims 11-14, wherein a bi-polar square wave amplitude is modulated at a video capture rate for the imaging system.

16. The liquid crystal assembly of any one of claims 13-15, wherein the amplitude of the bi-polar square wave is modulated from 500 to 2000 Hz.

17. The liquid crystal assembly of any one of claims 1-16, wherein the drive voltage source toggles the liquid crystal between the first voltage and the second voltage in durations from 1 milliseconds to 40 milliseconds inclusive, or 2 milliseconds to 60 milliseconds inclusive, 60 milliseconds to 100 milliseconds inclusive, and up to 300 milliseconds inclusive.

18. The liquid crystal assembly of any one of claims 1-17, wherein the drive voltage source toggles the liquid crystal between the first voltage and second voltage at a video rate sampling duration of approximately between 1 ms to 10 ms, between 2 ms and 20 ms, or 6 ms.

19. The liquid crystal assembly of any one of claims 1-18, wherein the liquid crystal is a twisted nematic (TN) liquid crystal.

20. The liquid crystal assembly of any one of claims 1-19, wherein the liquid crystal is heated to a range of 30° C. to 50° C.

21. The liquid crystal assembly of any one of claims 1-20, wherein the liquid crystal is enclosed in a housing of the imaging system and wherein the liquid crystal is heated by waste heat generated by other components of the imaging system.

22. The liquid crystal assembly of any one of claims 4-21, wherein a transition speed between the open state and the closed state is determined by a temperature of the liquid crystal and a voltage source drive level.

23. The liquid crystal assembly of any one of claims 1-22, wherein the polarizer is an engineered dyestuff polarizer.

24. The liquid crystal assembly of any one of claims 1-23, wherein the polarizer is configured to have a light cut-off in a range optimal for a fluorescent imaging process.

25. The liquid crystal assembly of any one of claims 1-24, wherein the polarizer is chosen to lose effectiveness or cutoff between 650 and 800 nm, or 600 nm and about 800 nm, or between about 700 nm to about 800 nm, about 800 nm to about 950 nm, about 800 nm to about 880 nm, about 775 nm to about 795 nm, or about 785 nm.

26. The liquid crystal assembly of any one of claims 1-25, wherein the polarizer is chosen to lose effectiveness or cutoff by 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm.

27. The liquid crystal assembly of any one of claims 1-26, further comprising a beam splitter.

28. The liquid crystal assembly of claim 27, wherein the beam splitter has a front surface and a back surface.

29. The liquid crystal assembly of claim 28, wherein the front surface has a front surface coating and the back surface has a back surface coating.

30. The liquid crystal assembly of claim 29, wherein the front surface coating has a higher P polarization reflection than the back surface coating.

31. The liquid crystal assembly of claim 30, wherein the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1.

32. The liquid crystal assembly of any one of claims 29-31, wherein S polarized reflections of the front surface and the back surface of the beam splitter are minimized by the front surface coating and the back surface coating.

33. The liquid crystal assembly of any one of claims 1-32, wherein the polarizer is configured to block or attenuate ghosting or secondary reflections reflected from a beam splitter.

34. The liquid crystal assembly of any one of claims 27-33, wherein the polarizer is configured to block S polarized light from the beam splitter.

35. The liquid crystal assembly of claims 27-34 wherein the polarizer is configured to allow P polarized light to pass through the liquid crystal assembly to an imaging lens and/or a camera when the liquid crystal is in the open state.

36. The liquid crystal assembly of any one of claims 27-35, wherein the first polarizer allows a front to back reflectivity ratio from the beam splitter of at least 2:1, 4:1, 6:1, 8:1, 10:1, 12:1, 15:1, 17:1, or 20:1 to pass through to an imaging lens and/or camera.

37. The liquid crystal assembly of any one of claims 27-36, wherein a total reflectivity from the beam splitter is about 11% or lower, about 10% or lower, about 9% or lower, about 8% or lower, about 7% or lower, about 6% or lower, about 5% or lower, about 4% or lower, or about 3% or lower.

38. The liquid crystal assembly of any one of claims 1-37, further comprising two polarizers or more.

39. The liquid crystal assembly of any one of claims 1-38, adapted to a medical device including a surgical microscope, confocal microscope, fluorescence scope, exoscope, endoscope, or surgical robot.

40. The liquid crystal assembly of claim 39, wherein the liquid crystal assembly is adapted to the medical device optionally through use of an optical gasket.

41. The liquid crystal assembly of claim 39-40, wherein the medical device is a KINEVO system (e.g., KINEVO 900), OMPI PENTERO system (e.g., PENTERO 900, PENTERO 800), or Leica FL800 system.

42. A method for imaging an emission light emitted by a fluorophore using the liquid crystal assembly of any one of claims 1-41.

43. A method for imaging an emission light emitted by a fluorophore at an imaging system comprising a liquid crystal assembly; the method comprising:
allowing or directing a visible light to a sample;
directing an excitation light to the sample;
directing the emission light and a reflected visible light from the sample to the liquid crystal assembly; wherein the liquid crystal assembly comprises:
an engineered polarizer disposed in a path of light from sample; the polarizer configured to have a light cut-off in a range between 600 nm and 900 nm;
a liquid crystal disposed in a path of light transmitted through the polarizer; and
a drive voltage source configured to apply a first voltage and a second voltage to the liquid crystal; wherein the first voltage and the second voltage toggle the liquid crystal between an open state and a closed state;
directing the emission light and the reflected visible light through the engineered polarizer, wherein the reflected visible light having a wavelength below the light cut-off passes through the polarizer with approximately 50% attenuation and the emission light having a wavelength above the light cut-off passes through the polarizer with minimal attenuation;
directing a polarized visible light and unpolarized emission light to the liquid crystal;
applying the first voltage to the liquid crystal to assume the open state for a first video rate sampling duration;
detecting the polarized light at an imaging sensor;
applying the second voltage to the liquid crystal to assume the closed state and block the polarized light from passing to the imaging sensor; and
detecting the unpolarized emission light at the imaging sensor during the closed state for a second video rate sampling duration.

44. The method of claim 43, wherein the minimal attenuation of the emission light having a wavelength above the light cut-off is 0% attenuation to 15% attenuation.

45. The method of claim 43 or 44, wherein the reflected visible light having a wavelength below the light cut-off passes through the polarizer at approximately 50% or less attenuation.

46. The method of any one of claims 43-45, wherein the first video rate sampling duration is between 1 ms to 10 ms, between 2 ms and 20 ms, or 6 ms.

47. The method of any one of claims 43-46, wherein the second video rate sampling duration is between 1 ms to 10 ms, between 2 ms and 20 ms, or 6 ms.

48. The method of any one of claims 43-47, wherein the first voltage and second voltage achieve high and low electric fields respectively across the liquid crystal.

49. The method of any one of claims 43-48, wherein the first voltage and the second voltage toggle the liquid crystal between an open state and a closed state.

50. The method of any one of claims 43-49, wherein the first voltage toggles the liquid crystal to the open state and the second voltage toggles the liquid crystal to the closed state, or vice versa.

51. The method of any one of claims 43-50, wherein one of the first voltage or the second voltage is 0 V.

52. The method of any one of claims 43-51, wherein the second voltage is a plurality of voltages comprising a bi-polar square wave with an average value of 0 V.

53. The method of any one of claims 43-52, wherein the first voltage, or the second voltage, or both the first voltage and the second voltage, comprises a plurality of voltages creating a bi-polar square wave with an average value of 0V.

54. The method of any one of claims 52-53, wherein the bi-polar square wave has an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

55. The method of any one of claims 52-54, wherein the second voltage is a bi-polar square wave having an amplitude of +/−0.1 to +/−5V p-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

56. The method of any one of claims 52-55, wherein a bi-polar square wave amplitude is modulated at a video capture rate for the imaging system.

57. The method of any one of claims 43-56, wherein the first voltage is a bi-polar square wave having an amplitude of +/−0.1 to +/−5 Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

58. The method of any one of claims 54-57, wherein the amplitude is modulated from 500 to 2000 Hz.

59. The method of any one of claims 43-58 further comprising:
heating the liquid crystal using waste heat from the imaging system.

60. The method of any one of claims 43-59, wherein the polarizer is a dyestuff polarizer.

61. The method of any one of claims 43-60, wherein the polarizer is configured to have a light cut-off in a range optimal for a fluorescent imaging process.

62. The method of any one of claims 43-61, wherein the polarizer in the liquid crystal assembly is configured to have a light cut-off in a range between about 600 nm and about 850 nm, or between about 700 nm to about 850 nm, about 800 nm to about 950 nm, about 800 nm to about 880 nm, about 775 nm to about 795 nm, or about 785 nm.

63. The method of any one of claims 43-62, wherein the polarizer in the liquid crystal assembly is chosen to lose effectiveness or cut off by 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm.

64. The method of any one of claims 43-63, wherein the polarizer in the liquid crystal assembly further comprises two polarizers or more.

65. The method of any one of claims 43-64, wherein the excitation light is engineered to enhance a signal to noise (SNR) of an NIR image such that a peak intensity and duration of the excitation light creates a controlled energy excitation that falls below a safety threshold.

66. The method of any one of claims 43-65, further comprising directing the emission light and the reflected visible light to a beam splitter prior to directing the emission light and the reflected light to the liquid crystal assembly.

67. The method of claim 66, wherein the beam splitter has a front surface and a back surface.

68. The method of claim 67, wherein the front surface has a front surface coating and the back surface has a back surface coating.

69. The method of claim 68, wherein the front surface coating has a higher P polarization reflection than the back surface coating.

70. The method of claim 69, wherein the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1.

71. The method of any one of claims 68-70, wherein S polarized reflections of the front surface and the back surface of the beam splitter are minimized by the front surface coating and the back surface coating.

72. The method of any one of claims 43-71, wherein the polarizer is configured to block or attenuate ghosting or secondary reflections reflected from a beam splitter.

73. The method of any one of claims 66-72 wherein the polarizer is configured to block S polarized light from the beam splitter.

74. The method of any one of claims 66-73, wherein the polarizer is configured to allow P polarized light to pass through the liquid crystal assembly to an imaging lens or a camera when the liquid crystal is in the open state.

75. The method of any one of claims 66-74, wherein the polarizer allows a front to back reflectivity ratio from the beam splitter of at least 2:1, 4:1, 6:1, 8:1, 10:1, 12:1, 15:1, 17:1, or 20:1 to pass through to an imaging lens and camera.

76. The method of any one of claims 66-75, wherein a total reflectivity from the beam splitter is about 11% or lower, about 10% or lower, about 9% or lower, about 8% or lower, about 7% or lower, about 6% or lower, about 5% or lower, about 4% or lower, or about 3% or lower.

77. A liquid crystal assembly comprising any feature described, either individually or in combination with any other features, in any configuration, as disclosed herein.

78. A method for imaging an emission light emitted by a fluorophore at an imaging system comprising a liquid crystal assembly comprising any feature described, either individually or in combination with any other features, in any configuration, as disclosed herein.

79. A liquid crystal assembly for use with an imaging system having a light source to provide an excitation light, the assembly comprising:
a first polarizer disposed in a path of light from the light source; the first polarizer configured to have a light cut-off in a range outside of its range of polarization;
a liquid crystal disposed in a path of light transmitted through the first polarizer;
a second polarizer disposed in a path of light transmitted through the liquid crystal; the second polarizer configured to have a light cut-off in a range outside of its range of polarization; and
a drive voltage source configured to apply a first voltage and a second voltage to the liquid crystal; wherein the first voltage and second voltage toggle the liquid crystal between an open state and a closed state.

80. The liquid crystal assembly of claim 79, wherein the first polarizer and the second polarizer are cross-polarized.

81. The liquid crystal assembly of claim 79 or claim 80, wherein the liquid crystal is in the open state when one of the first voltage or second voltage is applied to the liquid crystal.

82. The liquid crystal assembly of any one of claims 79-81, wherein one of the first voltage or second voltage is 0 V.

83. The liquid crystal assembly of any one of claims 79-82, wherein the liquid crystal is in the closed state when one of the first voltage or the second voltage is applied to the liquid crystal.

84. The liquid crystal assembly of any one of claims 79-83, wherein the second voltage is a bi-polar square wave.

85. The liquid crystal assembly of claim 84, wherein bi-polar square wave has an amplitude of +/−0.1 to +/−5Vp-p, +/−3 to +/−30Vp-p, +/−16 Vp-p, +/−24 Vp-p, or +/−30 Vp-p.

86. The liquid crystal assembly of any one of claims 79-85, the liquid crystal assembly further comprising:
an imaging lens in a path of light transmitted through the second polarizer; and
a camera in a path of light transmitted through the imaging lens.

87. The liquid crystal assembly of claim 86, wherein when the liquid crystal is in the open state the liquid crystal rotates the polarization of a polarized visible light transmitted through the first polarizer allowing the polarized visible light to pass through the second polarizer to the imaging lens at about 50% or less attenuation.

88. The liquid crystal assembly of claim 87, wherein when the liquid crystal is in the closed state the liquid crystal does not rotate the polarization of the polarized visible light and the polarized visible light transmitted through the first polarizer is blocked by the second polarizer.

89. The liquid crystal assembly of any one of claims 79-88, wherein the light cut-off range of the first polarizer and the second polarizer is where a polarizer extinction ratio is poor or minimal.

90. The liquid crystal assembly of any one of claims 86-89, wherein light outside the light cut-off range of the first polarizer and second polarizer passes through the first polarizer and the second polarizer to the imaging lens with no attenuation or a minimal attenuation.

91. The liquid crystal assembly of claim 90, wherein the minimal attenuation is 15% or less.

92. The liquid crystal assembly of any one of claims 79-91, wherein the light cut-off range of the first polarizer is about 700 nm to about 800 nm or about 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm.

93. The liquid crystal assembly of any one of claims 79-92, wherein the light cut-off range of the second polarizer is about 700 nm to about 800 nm or about 700 nm, 725 nm, 750 nm, 775 nm, 780 nm, 785 nm, 790 nm, 795 nm, 800 nm, 805 nm, 810 nm, 815 nm, 820 nm, 825 nm, 830 nm, 835 nm, 840 nm, 850 nm, 855 nm, 860 nm, 865 nm, 870 nm, 875 nm, 880 nm, 885 nm, 890 nm, 895 nm, or 900 nm.

94. The liquid crystal assembly of any one of claims 79-93, wherein the light cut-off range of the first polarizer and the second polarizer is above a longest wavelength of the visible light spectrum.

95. The liquid crystal assembly of any one of claims 79-94, wherein when the liquid crystal is in the open state the liquid crystal assembly is configured to view visible light.

96. The liquid crystal assembly of any one of claims 79-95, wherein when the liquid crystal is in the closed state the liquid crystal assembly is configured to block visible light.

97. The liquid crystal assembly of any one of claim 96, wherein the blocked visible light allows for fluorescent imaging from a fluorophore.

98. The liquid crystal assembly of claim 97, wherein the fluorophore provides an emission light emitted by the fluorophore.

99. The liquid crystal assembly of claim 97 or 98, wherein the excitation light is white light, NIR light, IR light, or any other type of excitation light.

100. The liquid crystal assembly of any one of claims 79-99, wherein the excitation light is a near infrared light provided by a laser diode.

101. The liquid crystal assembly of any one of claims 79-100, further comprising a beam splitter.

102. The liquid crystal assembly of claim 101, wherein the beam splitter has a front surface and a back surface.

103. The liquid crystal assembly of claim 102, wherein the front surface has a front surface coating and the back surface has a back surface coating.

104. The liquid crystal assembly of claim 103, wherein the front surface coating has a higher P polarization reflection than the back surface coating.

105. The liquid crystal assembly of claim 103 or 104, wherein the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1.

106. The liquid crystal assembly of any one of claims 103-105, wherein S polarized reflections of the front surface and the back surface of the beam splitter are minimized by the front surface coating and the back surface coating.

107. The liquid crystal assembly of any one of claims 79-106, wherein the first polarizer is configured to block or attenuate ghosting or secondary reflections reflected from a beam splitter.

108. The liquid crystal assembly of claim 101-107, wherein the first polarizer is configured to block S polarized light from the beam splitter.

109. The liquid crystal assembly of claims 101-108, wherein the first polarizer and the second polarizer are configured to allow P polarized light to pass through the liquid crystal assembly to an imaging lens or a camera when the liquid crystal is in the open state.

110. The liquid crystal assembly of any one of claims 101-109, wherein the first polarizer allows a front to back reflectivity ratio from the beam splitter of at least 2:1, 4:1, 6:1, 8:1, 10:1, 12:1, 15:1, 17:1, or 20:1 to pass through to an imaging lens and a camera.

111. The liquid crystal assembly of any one of claims 101-110, wherein a total reflectivity from the beam splitter is about 11% or lower, about 10% or lower, about 9% or lower, about 8% or lower, about 7% or lower, about 6% or lower, about 5% or lower, about 4% or lower, or about 3% or lower.

112. The liquid crystal assembly of any one of claim 86, wherein the imaging lens and camera are configured to view a fluorescent image using a video rate sampling duration of between 1 ms to 10 ms, between 2 ms and 20 ms, or 6 ms.

113. The liquid crystal assembly of any one of claim 112, wherein the fluorescent image has a reduced motion blur.

114. A method for imaging an emission light emitted by a fluorophore at an imaging system comprising a liquid crystal assembly, the method comprising:
allowing or directing a visible light to a sample;
directing an excitation light to the sample;
directing the emission light and a reflected visible light to the liquid crystal assembly; wherein the liquid crystal assembly comprises:
a first engineered polarizer in a path of light from the sample; the first engineered polarizer configured to have a light cut-off in a range between 600 nm and 900 nm or 800 nm to 880 nm;
a liquid crystal disposed in a path of light transmitted through the first engineered polarizer;
a second engineered polarizer in a path of light transmitted through the liquid crystal; the second engineered polarizer configured to have a light cut-off in a range between 600 nm and 900 nm or 800 nm to 880 nm, wherein the first engineered polarizer and second engineered polarizer are cross-polarized; and
a drive voltage source configured to apply a first voltage and a second voltage to the liquid crystal; wherein the first voltage and the second voltage toggle the liquid crystal between an open state and a closed state;
directing the emission light and the reflected visible light through the first engineered polarizer, wherein the first engineered polarizer polarizes the reflected visible light below the light cut-off range producing a polarized reflected visible light;
directing the emission light and the polarized reflected visible light through the liquid crystal;
applying the first voltage to the liquid crystal to assume the open state for a first video rate sampling duration;
rotating the polarization of the polarized reflected visible light using the liquid crystal in the open state;
directing the emission light and the polarized reflected visible light through the second engineered polarizer, wherein the second engineered polarizer allows the polarized reflected visible light to pass through the second engineered polarizer;
detecting a first portion of the polarized reflected visible light passing through the second engineered polarizer during the open state at an imaging sensor;
applying the second voltage to the liquid crystal to assume the closed state for a second video rate sampling duration;
passing the emission light and the polarized reflected visible light through the liquid crystal without rotating the emission light and the polarized reflected visible light;
blocking the polarized reflected visible light with the second engineered polarizer; and
detecting a portion of the emission light during the closed state at the imaging sensor.

115. The method of claim 114, the method further comprising blocking a secondary reflection of the reflected visible light using the first polarizer.

116. The method of claim 114 or 115, wherein the excitation light is provided to the sample via a laser diode.

117. The method of claim 116, the method further comprising:
turning the laser diode off to stop providing an excitation light;
providing the second voltage to the liquid crystal to assume the closed state for a third video rate sampling duration;
detecting a dark background image of the sample; and
subtracting the dark background image of the sample from the detected portion of the emission light to produce a fluorescent image.

118. The method of any one of claims 114-117, wherein the first video rate sampling duration is about 2 ms to about 10 ms.

119. The method of any one of claims 114-118, wherein the second video rate sampling duration is about 8 ms to about 16 ms.

120. The method of any one of claim 117, wherein the third video rate sampling duration is about 8 ms to about 16 ms.

121. The method of any one of claims 114-120, wherein one of the first voltage or the second voltage is about 0 V.

122. The method of any one of claims 114-121, wherein the second voltage is a bi-polar square wave.

123. The method of claim 122, wherein bi-polar square wave has an amplitude of +/−0.1 to +/−5Vp-p, +/−3 to +/−30Vp-p, +/−16 Vp-p, +/−24 Vp-p, or +/−30 Vp-p, or an amplitude between +/−16 V (32 Vp-p) and +/−30 V (60 Vp-p), at any value inclusive, or an amplitude between +/−30 V (60 Vp-p) and +/−100 V (200 Vp-p), at any value inclusive.

124. The method of claim 117, wherein the fluorescent image has a reduced motion blur.

125. The method of any one of claims 114-124, wherein the second video rate sampling duration and third video rate sampling duration allow a post processing by digital gain.

126. The method of any one of claims 114-125, the method further comprising orienting the first engineered polarizer to block an undesired polarized light.

127. The method of claim 117, wherein the second video rate sampling duration and third video rate sampling duration enhance a signal to noise ratio (SNR) and a contrast to noise ratio (CNR) of the fluorescent image.

128. The method of claim 127, wherein the enhanced CNR enables a fast frame rate.

129. The method of any one of claims 114-128, wherein the method provides real-time viewing of the reflected visible light and a fluorescent image.

130. The method of any one of claims 114-129, further comprising directing the emission light and the reflected light to a beam splitter prior to directing the emission light and the reflected light to the liquid crystal assembly.

131. The method of claim 130, wherein the beam splitter has a front surface and a back surface.

132. The method of claim 131, wherein the front surface has a front surface coating and the back surface has a back surface coating.

133. The method of claim 132, wherein the front surface coating has a higher P polarization reflection than the back surface coating.

134. The method of claim 132 or 133, wherein the front surface coating and the back surface coating produce a P polarized light front surface to back surface reflectivity ratio of at least 2:1, at least 4:1, at least 6:1, at least 8:1, at least 10:1, at least 12:1, at least 14:1, at least 16:1, at least 18:1, or at least 20:1.

135. The method of any one of claims 132-134, wherein S polarized reflections of the front surface and the back surface of the beam splitter are minimized by the front surface coating and the back surface coating.

136. The method of any one of claims 114-135, wherein the first polarizer is configured to block or attenuate ghosting or secondary reflections reflected from a beam splitter.

137. The method of any one of claims 130-136, wherein the first polarizer is configured to block S polarized light from the beam splitter.

138. The method of any one of claims 130-137, wherein the first polarizer and the second polarizer are configured to allow P polarized light to pass through the liquid crystal assembly to an imaging lens and/or a camera when the liquid crystal is in the open state.

139. The method of any one of claims 130-138, wherein the first polarizer allows a front to back reflectivity ratio from the beam splitter of at least 2:1, 4:1, 6:1, 8:1, 10:1, 12:1, 15:1, 17:1, or 20:1 to pass through to an imaging lens and a camera.

140. The method of any one of claims 130-139, wherein a total reflectivity from the beam splitter is about 11% or lower, about 10% or lower, about 9% or lower, about 8% or lower, about 7% or lower, about 6% or lower, about 5% or lower, about 4% or lower, or about 3% or lower.

141. A method of imaging an abnormal tissue, cancer, tumor, vasculature or structure in a sample from a subject, the method comprising producing an image of the vasculature or structure by imaging fluorescence using the imaging system of any one of claim 1-42 or 79-106.

142. A method of imaging an abnormal tissue, cancer, tumor, vasculature or structure in a sample from a subject in accordance with the method of any one of claim 43-78 or 107-140, the method comprising producing an image of the abnormal tissue, cancer, tumor, vasculature or structure by imaging fluorescence using an imaging system comprising a liquid crystal assembly.

143. The method of claim 141 or 142, wherein the fluorescence imaged is autofluorescence, a contrast or imaging agent, chemical agent, a radiolabel agent, radiosensitizing agent, photosensitizing agent, fluorophore, therapeutic agent, an imaging agent, a diagnostic agent, a protein, a peptide, a nanoparticle, or a small molecule, or any combination thereof or any combination thereof.

144. The method of any one of claims 141-143, wherein the fluorescence imaged is autofluorescence, a contrast or imaging agent, chemical agent, a radiolabel agent, radiosensitizing agent, photosensitizing agent, fluorophore, therapeutic agent, an imaging agent, a diagnostic agent, a protein, a peptide, a nanoparticle, or a small molecule, or any combination thereof.

145. The method of any one of claims 141-144, wherein the method further comprises administering a contrast or imaging agent to the subject.

146. A method of imaging an abnormal tissue, cancer, tumor, vasculature or structure in a fluorophore from a subject using the imaging system of any one of claim 1-42 or 79-106; the method comprising:
(a) administering a contrast or imaging agent to the subject; and
(b) producing an image of the abnormal tissue, cancer, tumor, vasculature or structure by imaging the contrast or imaging agent using an imaging system.

147. A method of imaging an abnormal tissue, cancer, tumor, vasculature or structure in a fluorophore from a subject in accordance with the method of any one of claim 43-78 or 107-140, the method further comprising:
(a) administering a contrast or imaging agent to the subject; and
(b) producing an image of the abnormal tissue, cancer, tumor, vasculature or structure by imaging the contrast or imaging agent using an imaging system.

148. The method of claim 146 or 147, wherein the contrast or imaging agent comprises a dye, a fluorophore, a fluorescent biotin compound, a luminescent compound, a chemiluminescent compound, or any combination thereof.

149. The method of claims 146-148, wherein the contrast or imaging agent further comprises a protein, peptide, amino acid, nucleotide, polynucleotide, or any combination thereof.

150. The method of claims 146-149, wherein the contrast or imaging agent further comprises tozuleristide.

151. The method of any one of claims 146-150, wherein the contrast or imaging agent absorbs a wavelength between from about 200 nm to about 900 nm.

152. The method of any one of claims 146-151, wherein the contrast or imaging agent comprises DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, rythrosine, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin, coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl(2-aminoethyl) sulfonamide), a bimane dye, bimane mercaptoacetic acid, an NBD dye, a QsY 35, or any combination thereof.

153. The method of any one of claims 146-152, wherein the administering comprises intravenous administration, intramuscular administration, subcutaneous administration, intraocular administration, intra-arterial administration, peritoneal administration, intratumoral administration, intradermal administration, or any combination thereof.

154. The method of any one of claims 146-153, wherein the imaging comprises tissue imaging, ex vivo imaging, intraoperative imaging, or any combination thereof.

155. The method of any one of claims 146-154, wherein the sample is in an in vivo sample, an in situ sample, an ex vivo sample, or an intraoperative sample.

156. The method of any one of claims 146-155, wherein the sample is an organ, an organ substructure, a tissue, or a cell.

157. The method of any one of claims 146-156, wherein the sample autofluoresces.

158. The method of claim 157, wherein autofluorescence of the sample comprises an ocular fluorophore, tryptophan, or protein present in a tumor or malignancy.

159. The method of any one of claims 146-158, wherein the method is used to visualize vessel flow or vessel patency.

160. The method of any one of claims 146-159, wherein the abnormal tissue, cancer, tumor, vasculature or structure comprises a blood vessel, lymph vasculature, neuronal vasculature, or CNS structure.

161. The method of any one of claims 146-160, wherein the imaging is angiography, arteriography, lymphography, or cholangiography.

162. The method of any one of claims 146-161, wherein the imaging comprises detecting a vascular abnormality, vascular malformation, vascular lesion, organ or organ substructure, cancer or diseased region, tissue, structure or cell.

163. The method of claim 162, wherein the vascular abnormality, vascular malformation, or vascular lesion is an aneurysm, an arteriovenous malformation, a cavernous malformation, a venous malformation, a lymphatic malformation, a capillary telangiectasia, a mixed vascular malformation, a spinal dural arteriovenous fistula, or a combination thereof.

164. The method of any one of claims 146-163, wherein an organ or organ substructure is brain, heart, lung, kidney, liver, or pancreas.

165. The method of any one of claims 146-164, further comprising performing surgery on the subject.

166. The method of claim 146-165, wherein the surgery comprises angioplasty, cardiovascular surgery, aneurysm repair, valve replacement, aneurysm surgery, arteriovenous malformation or cavernous malformation surgery, venous malformation surgery, lymphatic malformation surgery, capillary telangiectasia surgery, mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or diseased region, tissue, structure or cell of the subject, or any combination thereof.

167. The method of any one of claims 146-166, wherein the imaging comprises imaging a vascular abnormality, cancer or diseased region, tissue, structure, or cell of the subject after surgery.

168. The method of any one of claims 146-167, further comprising treating a cancer in the subject.

169. The method of any one of claims 146-168, further comprising repair of an intracranial CNS vascular defect, a spinal CNS vascular defect; peripheral vascular defects; removal of abnormally vascularized tissue; ocular imaging and repair; anastomosis; reconstructive or plastic surgery; plaque ablation or treatment or restenosis in atherosclerosis; repair or resection (including selective resection), preservation (including selective preservation), of vital organs or structures such as nerves, kidney, thyroid, parathyroid, liver segments, or ureters; identification and management (sometimes preservation, sometimes selective resection) during surgery; diagnosis and treatment of ischemia in extremities; or treatment of chronic wounds.
